(19) United States
(12) Patent Application Publication
(10) Pub. No.: US 2015/0336915 A1
(43) Pub. Date: Nov. 26, 2015

(54) STRUCTURES OF PROTEASOME INHIBITORS AND METHODS FOR SYNTHESIZING AND USE THEREOF

(71) Applicant: Pono Corporation, Honolulu, HI (US)

(72) Inventors: Jarred Roy Engelking, Honolulu, HI (US); Karl Milton Taft, Honolulu, HI (US)

(21) Appl. No.: 14/410,474

(22) PCT Filed: Jul. 1, 2013
(86) PCT No.: PCT/US2013/048966

§ 371 Date: Dec. 22, 2014

Related U.S. Application Data
(60) Provisional application No. 61/667,396, filed on Jul. 2, 2012.

(51) Int. Cl. C07D 281/00; C07D 245/02; C07D 225/02; C07D 255/02

(52) U.S. Cl. CPC C07D 281/00; C07D 255/02; C07D 245/02; C07D 225/02

(57) ABSTRACT

Disclosed herein are novel structures of proteasome inhibitors and methods for synthesizing and use thereof, including novel structures of proteasome inhibitors, such as syrbactins and its analogs, and methods for synthesizing them and using them for effective proteasome inhibition.

### RELATED CASES

This application claims the benefit of U.S. Provisional Application No. 61/667,396, filed on Jul. 2, 2012, the entire disclosure of which is incorporated by reference herein.

### BACKGROUND

1. Field

The present invention relates generally to novel structures of proteasome inhibitors and methods for synthesizing and use thereof. More particularly, the present invention relates to novel structures of proteasome inhibitors, such as syrbactins and its analogs, and methods for synthesizing them and using them for effective proteasome inhibition.

2. Description of Related Art

Proteasome inhibitors, unlike other therapeutic compositions, are a class of promising inhibitors that distinguish between cancerous and normal cells. In other words, proteasome inhibitors appear to be more effective and active in cancer cells compared to normal cells. More than cancer, proteasome inhibitors are also effective in treatment of other diseases and pathological conditions.

A wide range of cellular substrates and processes are controlled by or affected by the ubiquitin-proteasome pathway. As a result, the ability of natural products and other compounds to act as proteasome inhibitors has attracted significant interest.

### SUMMARY

Intracellular protein turnover is crucial to maintenance of normal cellular homeostasis. Proteasome inhibitors are thought of as potential drug candidates due to their ability to induce programmed cell death, preferentially, in transformed cells (as compared to normal cells). The ubiquitin-proteasome pathway has emerged as a primary target for cancer therapy and led to the approval of one of the first protesome inhibitors, bortezomib, for relapsed/refractory multiple myeloma and mantle cell lymphoma. However, there still exist problems with patients developing refractoriness to such drugs as well as development of bortezomib-resistant (or other proteasome inhibitors) disease and possibly in a broader spectrum of diseases. As such, new proteasome inhibitor compositions are needed to continue to provide clinically valuable control of various diseases in which proteasome inhibitions leads to therapeutic efficacy.

There are therefore provided herein, in several embodiments, proteasome inhibitors comprising a core ring structure selected from a group consisting of a first structure (Formula I), a second structure (Formula II), and a third structure (Formula III) which are respectively,

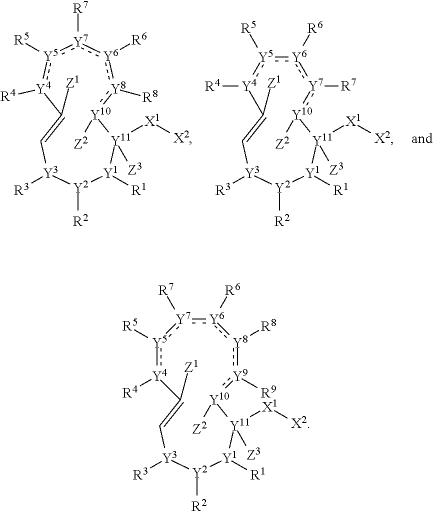

In several embodiments, Y¹ is at least one member selected from a group consisting of nitrogen, NH, oxygen, OH, sulfur, SO, SO₂, and carbon. In several such embodiments, each of Y², Y⁴, and Y⁶ is at least one member selected from a group consisting of nitrogen, NH, oxygen, OH, sulfur, SO, SO₂, CO, and carbon. In some embodiments, X¹ is absent or alternatively is at least one member selected from a group consisting of hydrogen, OH, CH₂O, COH, CO₂H, halide, NH, S, P(X²)₃, BOH, B(OH)₂, aryl, carbocycle, substituted aryl, substituted carbocycle, heterocycle, substituted heterocycle, alkyl, substituted alkyl, alkenyl, alkenyl substituted, alkynyl, alkynyl substituted, aralkyl, (CH₂CH₂Y¹³)ᵣ, JAJ, an amino-acid-based moiety, and (Y¹²R¹⁰LQR¹¹)_{q} (and each of q and r is an integer value between 1 and 10). In several embodiments, each of Y³, Y⁵, Y⁷, Y⁸, Y⁹, Y¹⁰, Y¹¹, Y¹², and Y¹³ is a moiety; and each of X², R¹, R², R³, R⁴, R⁵, R⁶, R⁷, R⁸, R⁹, R¹⁰, R¹¹, Z¹, Z², Z³, A, J, L, and Q is a moiety or absent.

In several embodiments, each of Y³, Y⁵, Y⁷, Y⁸, Y⁹, Y¹⁰, and Y¹¹ is at least one member selected from a group consisting of nitrogen, NH, oxygen, OH, sulfur, SO, SO₂, CO and carbon. In several embodiments, X¹ is hydrogen and X² is absent.

In additional embodiments, X² is absent or at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, (CH₂CH₂Y¹³)ᵣ, JAJ, and an amino-acid-based moiety.

In several embodiments, each of R¹, R², R³, R⁴, R⁵, R⁶, R⁷, R⁸, and R⁹ is absent or alternatively is at least one member selected from a group consisting of X¹, hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, halo, a heteroatom, and an amino-acid-based moiety.

In several embodiments, R¹⁰ is absent or at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, (CH₂CH₃Y¹³)ᵣ, JAJ, and an amino-acid-based moiety. In several such embodiments, A is at least one member selected from a group consisting of C═=O, C═=S, SO, and SO₂, and J is absent or at least one member selected from a group consisting of oxygen, sulfur, NH, and N-alkyl.

In several embodiments, R¹¹ is absent or at least one member selected from a group consisting of hydrogen, CF³, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, (CH₂CH₂Y¹³)ᵣ, JAJ, R¹²JAJ-alkyl-, R¹⁵J-alkyl-, (R¹²O)(R¹³O)P(═=O)O-alkyl-JAJJAJ-alkyl-, R¹² JAJ-alkyl-JAJJAJ-alkyl-, JAJheterocyclylMJAJ-alkyl-, (R¹²O)(R¹³O)P(═=O)O-alkyl-, (R¹⁴)₂N— alkyl-, (R¹⁴)₃N⁺— alkyl-, heterocyclylJ-, carbocyclylJ-, R¹⁵SO₂alkyl-, and R¹⁵SO₂NH, and each of R¹² and R¹³ is at least one member selected from a group consisting of hydrogen, metal cation, aryl, carbocycle, substituted aryl, substituted carbocycle, heterocycle, substituted heterocycle, alkyl, substituted alkyl, alkenyl, substituted alkenyl, alkynyl, substituted alkynyl, and aralkyl.

In several embodiments, R¹⁴ is at least one member selected from a group consisting of hydrogen and alkyl and R¹⁵ is at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, and aralkyl. In several embodiments, M is absent or alkyl. In several such embodiments, A is at least one member selected from a group consisting of C═=O, C═=S, SO, and SO₂, and J is absent or at least one member selected from a group consisting of oxygen, sulfur, NH, and N-alkyl. Also, in several embodiments, each of R¹, R², R³, R⁴, R⁵, R⁶, R⁷, R⁸, and R⁹ is absent or at least one member selected from a group consisting of X¹, hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, halo, a heteroatom, and an amino-acid-based moiety. In some embodiments, R¹⁰ and R¹¹ together form a ring that is at least one member selected from a group consisting of alkyl, substituted alkyl, and aralkyl. In additional embodiments, R¹² and R¹³ may together form a ring that is at least one member selected from a group consisting of alkyl, substituted alkyl, and aralkyl.

In several embodiments, each of Z¹, Z², and Z³ is absent or at least one member selected from a group consisting of hydrogen and fluorine.

In several embodiments, L is at least one member selected from a group consisting of C═=O, C═=S, SO, and SO₂.

In several embodiments, Q is absent or at least one member selected from a group consisting of carbon, oxygen, NH, and N-alkyl.

In several embodiments, Formula I is one member selected from a group consisting of a first structure, a second structure, a third structure, a fourth structure, a fifth structure, and a sixth structure, and said first structure is represented by:

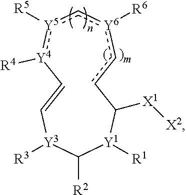

said second structure is represented by:

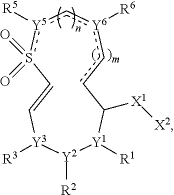

said third structure is represented by:

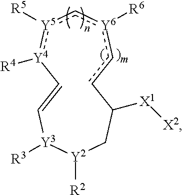

said fourth structure is represented by:

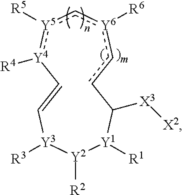

said fifth structure is represented by:

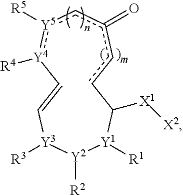

and
said sixth structure is represented by:

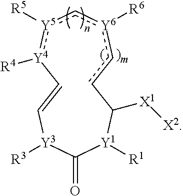

In several embodiments, X³ is at least one member selected from a group consisting of oxygen, sulfur, SO, SO₂, CO, and carbon; and, CH₂O, COH, CO₂H, halide, P(X²)₃, BOH, B(OH)₂, aryl, carbocycle, substituted aryl, substituted carbocycle, heterocycle, substituted heterocycle, alkyl, substituted alkyl, alkenyl, alkenyl substituted, alkynyl, alkynyl substituted, aralkyl, (CH₂CH₂Y¹³)ᵣ, JAJ, an amino-acid-based moiety, and (Y¹²R¹⁰LQR¹¹)_{q}, and each of q and r is an integer value between 1 and 10; and each of n and m is an integer value equal to 0, 1, or 2. In several embodiments, each of n and m equals 1. In additional embodiments, n equals 0 and m equals 1. In still further embodiments, n equals 1 and m equals 2.

In several embodiments, said Formula I is a core ring structure selected from a group consisting of a first structure, a second structure, a third structure, a fourth structure, a fifth structure, a sixth structure, a seventh structure, an eighth structure and a ninth structure, said first structure is represented by:

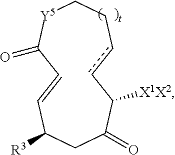

said second structure is represented by:

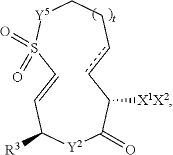

said third structure is represented by:

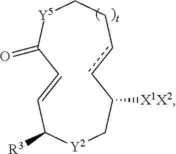

said fourth structure is represented by:

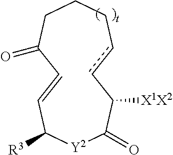

said fifth structure is represented by:

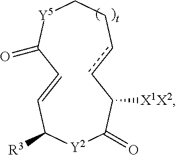

said sixth structure is represented by:

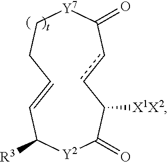

said seventh structure is represented by:

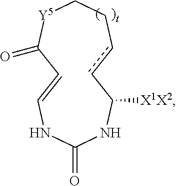

said eighth structure is represented by:

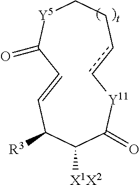

said ninth structure is represented by:

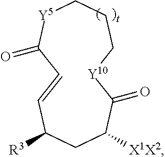

and
wherein t is an integer value between 0 and 2.

In several embodiments, there are additional provided methods of synthesizing a proteasome-inhibiting core structure, comprising: coupling a vinyl amino acid and an amino alcohol to produce vinyl functionalized compound, coupling said vinyl functionalized compound with a phosphonate compound to produce a reactive precursor, phosphonate compound is produced by coupling a phosphonate precursor and a 1-butene derivative, oxidizing said reactive precursor to yield an aldehyde-based proteasome-inhibiting precursor; and cyclizing said aldehyde-based proteasome-inhibiting precursor using a coupling reaction to produce a proteasome-inhibiting core structure.

In several embodiments, the vinyl amino acid is represented by the following formula:

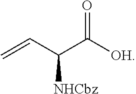 [SMILES: C=C[C@H](NC(=O)OCc1ccccc1)C(=O)O]

In several embodiments, the amino alcohol is represented by the formula:

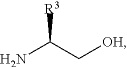

and in certain such embodiments, R³ is absent or a moiety.

In several embodiments, the coupling of said vinyl amino acid includes a peptide coupling reaction. In several embodiments, the coupling of said vinyl functionalized compound includes a cross-metathesis reaction. In certain such embodiments, the cross-metathesis reaction is carried out in the presence of an olefin metathesis catalyst.

In several embodiments, the vinyl functionalized compound is represented by a formula:

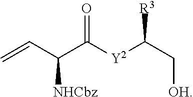

In certain such embodiments, R³ is absent or a moiety, and Y² is at least one member selected from a group consisting of nitrogen, NH, oxygen, OH, sulfur, SO, SO₂, CO, and carbon.

In several embodiments, the carrying out includes a nucleophilic substitution.

In several embodiments, the phosphonate compound is represented by a formula:

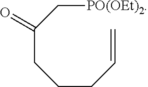

In several embodiments, the proteasome inhibitors disclosed herein (or synthesized by the methods herein can trigger apoptosis in proliferating cells (such as for example, cancer cells) based on promotion and/or suppression of positive and negative regulators of cell growth.

In some embodiments, the proteasome inhibitors disclosed herein are administered to a subject receiving a therapy such as, for example, inhibition of antigen presentation, anticancer therapies, antiviral therapies, anti-inflammatory therapies, and anti-bacterial therapies. Diseases or symptoms that can be treated include, but are not limited to, tissue or organ transplant rejection, autoimmune diseases, Alzheimer's disease, amyotropic lateral sclerosis, asthma, cancer, autoimmune thyroid disease, type I diabetes, ischemia-reperfusion injury, cachexia, graft rejection, hepatitis B, inflammatory bowel disease, sepsis, measles, subacute sclerosing panencephalitis (SSPE), mumps, parainfluenza, malaria, human immunodeficiency virus diseases, simian immunodeficiency viral diseases, Rous sarcoma viral diseases, cerebral ischemic injury, ischemic stroke, inflammation, inflammatory disease and tuberculosis. In addition, in several embodiments the proteasome inhibitors disclosed herein are used to immunize a subject that can be at risk of developing an infectious disease or tumor.

When used in treating diseases, such as those disclosed herein, the proteasome inhibitors can be administered to a subject in singular or sequential doses. Sequential doses can be of the same volume and/or concentration, or may be serially increased, serially decreased, or adjusted based on specific patient characteristics. Sequential doses can be separated from one another by various time periods, e.g., hours, days, weeks, etc. In several embodiments, continuous dosing is employed (e.g., intravenous drip). Depending on the embodiment, other dosing routes (e.g., intramuscular, subcutaneous, intrarterial, intraperitoneal, intracerebrospinal, subcutaneous, intra-articular, intrasynovial, intrathecal, oral, rectal, topical or nasal or oral inhalation routes) are used. Oral dosing (e.g., by liquid, capsule, pill etc.) is used in some embodiments. An effective amount of a therapeutic agent to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 100 mg/kg or more, depending on the factors mentioned above. Typically, the clinician will administer an amount until a dosage is reached that provides the required biological effect. The progress of this therapy can be monitored, e.g., by conventional assays.

### BRIEF DESCRIPTION OF THE DRAWINGS

FIG. 1 is an illustration that depicts the chemical structure of currently known syrbactin compounds, i.e., syringolin A, syringolin B and glidobactin A cepafungin II.

FIG. 2 is an illustration that depicts the chemical structure of inventive proteasome inhibiting compounds, according to one embodiment of the present invention.

FIG. 3 is an illustration that depicts the chemical structure of inventive proteasome inhibiting core structures, according to one embodiment of the present invention.

FIG. 4A is an illustration that depicts the chemical structure of inventive proteasome inhibiting core structures, according to certain preferred embodiments of the present invention.

FIG. 4B is an illustration that depicts the chemical structure of inventive proteasome inhibiting core structures, according to other preferred embodiments of the present invention.

FIG. 5 is an illustration that depicts the chemical structure of inventive ligand structures, according to certain embodiments of the present invention.

FIG. 6 is an illustration that depicts a synthesis pathway, according to one embodiment of the present invention, of proteasome inhibiting core structures.

FIG. 7 is an illustration that depicts a synthesis pathway, according to one embodiment of the present invention, of a proteasome inhibitor formed using the cores structure of FIG. 6.

FIG. 8 is an illustration that depicts a synthesis pathway, according to another embodiment of the present invention, of proteasome inhibiting core structures.

FIG. 9 is an illustration that depicts a synthesis pathway, according to another embodiment of the present invention, of a proteasome inhibiting core-ligand precursor formed using the cores structure of FIG. 8.

FIG. 10 is an illustration that depicts a synthesis pathway, according to yet another embodiment of the present invention, of proteasome inhibiting core structures.

FIG. 11 is an illustration that depicts a synthesis pathway, according to yet another embodiment of the present invention, of a proteasome inhibiting core-ligand precursor formed using the cores structure of FIG. 10.

FIG. 12 is an illustration that depicts a synthesis pathway, according to yet another embodiment of the present invention, of proteasome inhibiting core structures.

FIG. 13 is an illustration that depicts a synthesis pathway, according to yet another embodiment of the present invention, of a proteasome inhibiting core-ligand precursor formed using the cores structure of FIG. 12.

FIG. 14 is an illustration that depicts a synthesis pathway, according to yet another embodiment of the present invention, of proteasome inhibiting core structures.

FIG. 15 is an illustration that depicts a synthesis pathway, according to yet another embodiment of the present invention, of a proteasome inhibiting core-ligand precursor formed using the cores structure of FIG. 14.

FIG. 16 is an illustration that depicts a synthesis pathway, according to yet another embodiment of the present invention, of proteasome inhibiting core structures.

FIG. 17 is an illustration that depicts a synthesis pathway, according to yet another embodiment of the present invention, of a proteasome inhibiting core-ligand precursor formed using the cores structure of FIG. 16.

FIG. 18 is an illustration that depicts a synthesis pathway, according to yet another embodiment of the present invention, of proteasome inhibiting core structures.

FIG. 19 is an illustration that depicts a synthesis pathway, according to yet another embodiment of the present invention, of a proteasome inhibiting core-ligand precursor formed using the cores structure of FIG. 18.

FIG. 20 is an illustration that depicts a synthesis pathway, according to yet another embodiment of the present invention, of proteasome inhibiting core structures.

FIG. 21 is an illustration that depicts a synthesis pathway, according to yet another embodiment of the present invention, of proteasome inhibiting core structures.

FIG. 22 is an illustration that depicts a synthesis pathway, according to yet another embodiment of the present invention, of a proteasome inhibitor.

FIG. 23 is an illustration that depicts a synthesis pathway, according to yet another embodiment of the present invention, of a proteasome inhibiting core-ligand precursor.

FIG. 24 is an illustration that depicts a synthesis pathway, according to yet another embodiment of the present invention, of a proteasome inhibitor.

FIG. 25 is an illustration that depicts a synthesis pathway, according to yet another embodiment of the present invention, of a proteasome inhibiting core-ligand precursor.

FIG. 26 is an illustration that depicts a synthesis pathway, according to yet another embodiment of the present invention, of a proteasome inhibiting core-ligand precursor.

FIG. 27 is an illustration that depicts a synthesis pathway, according to yet another embodiment of the present invention, of a proteasome inhibiting core-ligand precursor.

FIG. 28 is an illustration that depicts a synthesis pathway, according to yet another embodiment of the present invention, of a proteasome inhibiting core-ligand precursor.

FIG. 29 is an illustration that depicts a synthesis pathway, according to yet another embodiment of the present invention, of a proteasome inhibiting core-ligand precursor.

FIG. 30 is an illustration that depicts synthesis pathways, according to other embodiments of the present invention, of ligand intermediates and a saturated acid intermediate.

FIG. 31 is an illustration that depicts a synthesis pathway, according to yet another embodiment of the present invention, of a proteasome-inhibiting core with ligand.

FIG. 32 is an illustration that depicts pathways, according to other embodiments of the present invention, of attaching a ligand to proteasome-inhibiting core structures.

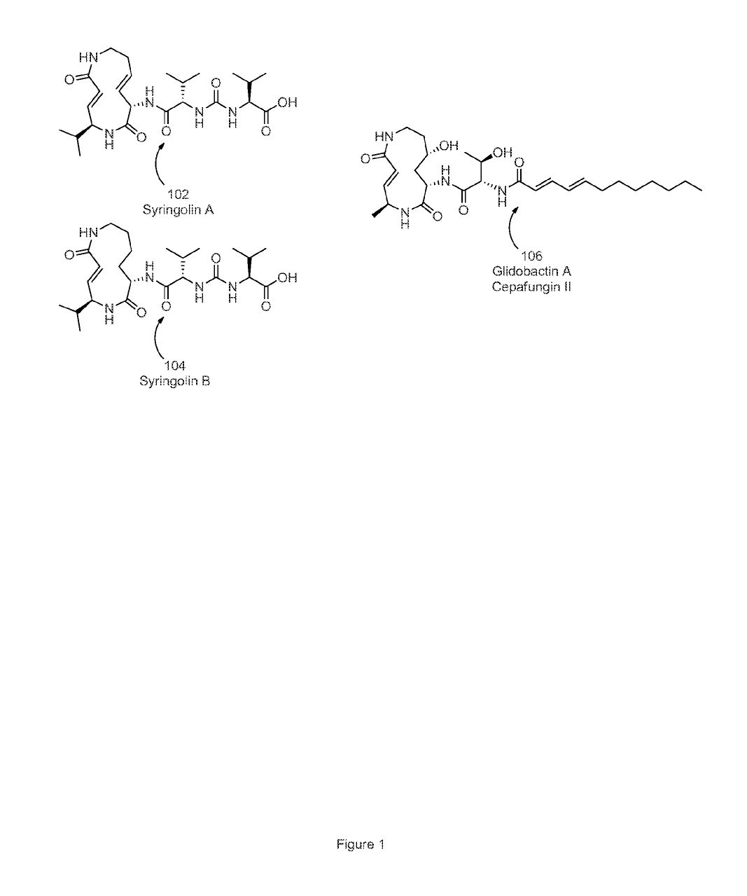

Those of skill in the art understand that the drawings, described below, are for illustrative purposes only. The drawings are not intended to limit the scope of the present teachings in any way.

### DETAILED DESCRIPTION

Proteasome inhibitors represent are a class of inhibitors with a wide variety of potential clinical applications, such as, for example, the treatment of cancer and many other pathological and autoinflammatory diseases. By way of example, proteasome inhibitors induce multiple myeloma (MM) cell apoptosis. Multiple myeloma (MM) is a malignancy of the bone marrow which causes cancerous plasma cells to uncontrollably grow and create tumors in multiple sites. Normally, plasma cells account for less than five percent of the cells in bone marrow. In those individuals, who suffer from MM, however, plasma cells account for anywhere from ten percent to more than ninety percent of the cells in the bone marrow. Over time, the abnormal cells can permeate the interior of the bone and erode the bone cortex (outer layer). These weakened bones are more susceptible to bone fractures, especially in the spine, skull, ribs, and pelvis. The annual incidence of MM is approximately 4 per 100,000 people, and the condition is particularly common in the elderly population with a median age of 65 years; only 3% of patients with MM are less than 40 years old.

Proteasome inhibitors are believed to be effective in the treatment of MM because they inducing a stress response in MM cells contributing to apoptosis. Proteasomes (also referred to as multicatalytic protease (MCP), multicatalytic proteinase, multicatalytic proteinase complex, multicatalytic endopeptidase complex, 20S, 26S, or ingensin) are a large, multiprotein complex present in both the cytoplasm and the nucleus of all eukaryotic cells. It is a highly conserved cellular structure that is responsible for the ATP-dependent proteolysis of most cellular protein. The 26S proteasome consists of a 20S core catalytic complex that is capped at each end by a 19S regulatory subunit. The 26S proteasome is able to degrade proteins that have been marked by the addition of ubiquitin molecules. Proteasome inhibitors, in particular those in accordance with the compositions and methods disclosed herein, which inhibit proteasome activity, may arrest or delay cancer progression by interfering with the ordered degradation of cell cycle proteins and/or tumor suppressors.

Bortezomib, also known as PS-341 or [(1R)-3-methyl-1-({(2S)-3-phenyl-2-[(pyrazin-2-ylcarbonyl)amino]propanoyl-}amino)butyl]boronic acid, is a boronic acid dipeptide proteasome inhibitor that has shown anti-tumor activity both in vitro and in clinical trials involving MM patients. In addition to bortezomib, other proteasome inhibitors are also known. For example, a group of novel boronic acid proteasome inhibitors, including the compound known as CEP-18770. CEP-18770, whose chemical name is [(1R)-1-[[(2S,3R)-3-hydroxy-246-phenyl-pyridine-2-carbonyl)amino]-1-oxobutyl]amino]-3-methylbutylboronic acid, have been shown to be orally active and have a favorable tumor selectivity profile for the treatment of MM and other malignancies responsive to proteasome inhibition.

Unfortunately, use of prolonged Bortezomib therapy or treatment using novel boronic acid proteasome inhibitors can lead to drug resistance in patients. In other words, although patients may initially respond to chemotherapy and/or steroids, most ultimately suffer from the disease when it has become resistant to treatment. As a result, for patients who progress after primary chemotherapy, which may also involve autologous stem cell transplantation, further chemotherapy is generally of limited benefit. Overall, the results of conventional cytotoxic chemotherapy, at least to date, for MM suggests that a plateau is reached and patients become refractory to treatment.

Furthermore, drug compositions currently employed during treatment offer core structures without an appreciable diversity in the side chains. As a result, a limited pool of proteasome inhibiting compositions are current available to carry out drug development for cancer and other malignancies. What is, therefore, needed are alternative treatment options that can offer the best long-term outcome for cancer (e.g., MM) patients and those that suffer from other malignancies. The need is especially urgent for novel therapies for patients with relapsed or refractory disease and who are typically more symptomatic and may be older with potential comorbidities and are especially challenging to treat. Accordingly, several embodiments disclosed herein provide for chemical structures that (i) modify the core structure in order to increase the reactivity of certain active portions of the core, (ii) modify the core structure to promote steric interaction with the target proteasome (or subunit thereof) and/or (iii) modify the ligand (e.g., the tail) structure and/or position to enhance the interaction of the compound with the target proteasome (or subunit thereof). Surprisingly, in several embodiments, these alterations lead to increased potency, therapeutic efficacy, specificity, and/or reduced side effects.

In the following description, numerous specific details are set forth in order to provide a thorough understanding of the present invention. It will be apparent, however, to one skilled in the art that the present invention is practiced without limitation to some or all of these specific details. In other instances, well-known process steps have not been described in detail in order to not unnecessarily obscure the invention.

As used herein, a the term “subject” shall be given its ordinary meaning and shall also include any organism, including an animal, for which diagnosis, screening, monitoring or treatment is contemplated. Animals include mammals such as primates and domesticated animals. In several embodiments, the primate is a human. A patient refers to a subject such as a mammal, primate, human or livestock subject afflicted with a disease condition or for which a disease condition is to be determined or risk of a disease condition is to be determined.

As used herein, the term “cancer” and “cancerous” shall be given their ordinary meanings and shall also refer to or describe the physiological condition in mammals that is typically characterized by unregulated cell growth. Examples of cancer include, but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, head and neck cancer, ovarian cancer and neuroblastoma. While the term “cancer” as used herein is not limited to any one specific form of the disease, it is believed that the methods of the invention can be effective for cancers which are found to be blood-related cancers and those cancers in which solid tumors form, including, but not limited to, multiple myeloma, mantle cell lymphoma and leukemias. Additionally, cancerous tissues that can be treated with the compositions disclosed herein include, but are not limited to acute lymphoblastic leukemia (ALL), acute myeloid leukemia (AML), adrenocortical carcinoma, Kaposi's sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma, breast cancer, bronchial tumors, Burkitt's lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin's lymphoma, non-Hodgkin's lymphoma, hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer. Further non-limiting examples of potential diseases that can be treated, methods of treatment, and other compounds that can be used or modified for use with those disclosed herein can be found in U.S. Pat. Pub. No. 2010/0267070 A1 to Bachmann et al., which is hereby incorporated by reference in its entirety.

In embodiments of the invention, the compounds of the invention can be administered as the sole active agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound disclosed herein with at least one other pharmaceutical agent, is intended to embrace administration of each agent in a sequential manner in a regimen that will provide beneficial effects of the drug combination, and is intended as well to embrace co-administration of these agents in a substantially simultaneous manner, such as in a single dose having a fixed ratio of these active agents or in multiple, separate doses for each agent.

Specifically, the administration of the compounds disclosed herein can be in conjunction with additional therapies known to those skilled in the art in the prevention or treatment of neoplastic disease, such as with radiation therapy or with cytostatic or cytotoxic agents.

Standard treatment of primary tumors can consist of surgical excision followed by either radiation or intravenously (IV) administered chemotherapy. The typical chemotherapy regime consists of either DNA alkylating agents, DNA intercalating agents, CDK inhibitors, or microtubule poisons. The chemotherapy doses used are just below the maximal tolerated dose and therefore dose limiting toxicities typically include, nausea, vomiting, diarrhea, hair loss, neutropenia and the like.

A large number of antineoplastic agents is available in commercial use, in clinical evaluation and in pre-clinical development, which can be selected for treatment of neoplastic disease by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents which can be used in combination with embodiments of the invention disclosed herein comprises antimetabolite-type/thymidilate synthase inhibitor antineoplastic agents. Suitable antimetabolite antineoplastic agents can be selected from, but are not limited to, the group consisting of 5-FU-fibrinogen, acanthifolic acid, aminothiadiazole, brequinar sodium, cammofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2¹-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

A second family of antineoplastic agents which can be used in combination with embodiments of the invention disclosed herein comprises alkylating-type antineoplastic agents. Suitable alkylating-type antineoplastic agents can be selected from, but not limited to, the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumitomo DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unfitted G-6-M, Chinoin GYKI-17230, hepsul-fam, Ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which can be used in combination with embodiments of the invention disclosed herein comprises antibiotic-type antineoplastic agents. Suitable antibiotic-type antineoplastic agents can be selected from, but are not limited to, the group consisting of Taiho 4181-A, aclarubicin, actinomycin D, actinoplanone, Erbamont ADR-456, aeroplysinin derivative, Ajinomoto AN-201-1, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, crbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, zorubicin, peptide boronates (e.g. bortezomib), α′β′-epoxyketones (e.g. epoxomoxin), β-lactones (e.g. salinosporamide A, salinosporamide B, fluorosalinosporamide, lactacystin), cinnabaramide A, cinnabaramide B, cinnabaramide C, belactosines (e.g. homobelactosin C), fellutamide B, TMC-95A, PS-519, omuralide, and antiprotealide ‘Salinosporamide-Omularide Hybrid.’

A fourth family of antineoplastic agents which can be used in combination with embodiments of the invention disclosed herein comprises a miscellaneous family of antineoplastic agents, including, but not limited to, tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes C1H-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine; cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, lnvitron protease nexin 1, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, with anolides and Yamanouchi YM-534.

In some embodiments, the compounds disclosed herein can be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, RAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alpha, daunorubicin, doxorubicin, tretinoin, edelfo sine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alpha, interferon alpha, natural, interferon alpha-2, interferon alpha-2a, interferon alpha-2b, interferon alpha-N1, interferon alpha-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alpha-2b, pentosan polysulfate sodium, pentostatin, picibanil, pirarubicin, rabbit antithymocyte polyclonal antibody, polyethylene glycol interferon alpha-2a, porfimer sodium, raloxifene, raltitrexed, rasburicase, rhenium Re 186 etidronate, RII retinamide, rituximab, romurtide, samarium (153 Sm) lexidronam, sargramostim, sizofuran, sobuzoxane, sonermin, strontium-89 chloride, suramin, tasonermin, tazarotene, tegafur, temoporfin, temozolomide, tenipo side, tetrachlorodecaoxide, thalidomide, thymalfasin, thyrotropin alpha, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SDO1 (Amgen), fulvestrant, galocitabine, gastrin 17-immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb) (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), valspodar, or proteasome inhibitors, including, but not limited to, peptide aldehydes (such as, for example, calpain inhibitor I/II, MG132), peptide boronates (such as, for example, Velcade/bortezomib, CEP-18770), β-lactones (such as, for example, lactacystin, Salinosporamide A/B, NPI-0052), peptide vinyl sulfones (such as, for example, NLVS, YLVS, ZLVS), and peptide epoxylketones (such as, for example, epoxomycin, TMC, carfilzomib).

In some embodiments, the compounds disclosed herein can be used in co-therapies with other agents, such as other kinase inhibitors including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, NSAID's, SOD mimics or αvβ3 inhibitors, and anti-inflammatories.

In some embodiments, the combinations disclosed herein can comprise a therapeutically effective amount that provides additive or synergistic therapeutic effects. The combination of at least one proteasome inhibiting compound plus a second agent described herein can be useful for synergistically enhancing a therapeutic response, such as, for example, inducing apoptosis in malignant cells, reducing tumor size, or providing chemoprevention. Such combinations can be administered directly to a subject for preventing further growth of an existing tumor, enhancing tumor regression, inhibiting tumor recurrence, or inhibiting tumor metastasis. The combinations can be provided to the subject as immunological or pharmaceutical compositions. In addition, components of the synergistic combination can be provided to the subject simultaneously or sequentially, in any order.

In some embodiments, synergistic combinations of compounds, and methods of using the same, can prevent or inhibit the growth of a tumor or enhance the regression of a tumor, for instance by any measurable amount. The term “inhibit” does not require absolute inhibition. Similarly, the term “prevent” does not require absolute prevention. Inhibiting the growth of a tumor or enhancing the regression of a tumor includes reducing the size of an existing tumor. Preventing the growth of a tumor includes preventing the development of a primary tumor or preventing further growth of an existing tumor. Reducing the size of a tumor includes reducing the size of a tumor by a measurable amount, for example at least 5%, at least 10%, at least 20%, at least 30%, at least 40%, at least 50%, at least 60%, at least 70%, at least 80%, at least 85%, at least 90%, at least 95%, at least 99%, or 100%.

The eukaryotic 20S proteasome contains three catalytic subunits (β1, β2, and (β5) conferring caspase-like, trypsin-like and chymotrypsin-like proteolytic activities, respectively. In some embodiments, compounds such as those disclosed herein, can be administered to a subject in an amount effective to reversibly or irreversibly inhibit one, two, or more of the aforementioned catalytic subunits described above.

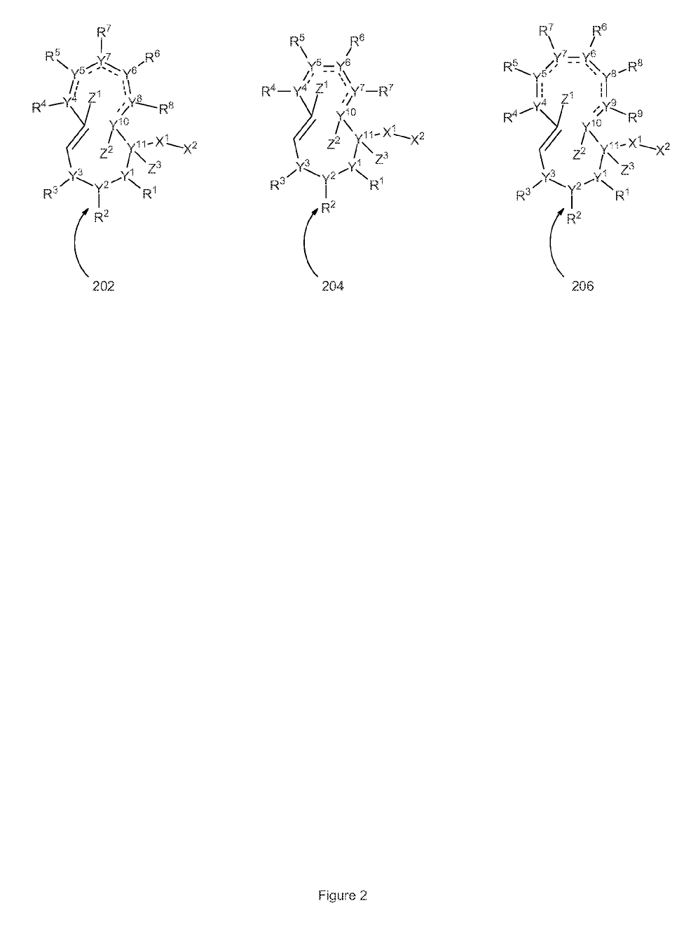

In several embodiments, proteasome inhibitors comprise an 11-13 membered ring core, such as an 11, 12, or 13 membered ring core. FIG. 2 describes non-limiting examples of proteasome inhibitors according to certain embodiments of the present invention, having core structures denoted by reference numerals 202, 204, and 206. In certain such embodiments, Y¹—Y¹¹ is selected from Nitrogen, NH, Oxygen, OH, Sulfur, SO, SO₂, CO, or Carbon; and X¹ is absent or at least one member selected from a group consisting of hydrogen, OH, CH₂O, COH, CO₂H, halide, NH, S, P(X²)₃, BOH, B(OH)₂, aryl, carbocycle, substituted aryl, substituted carbocycle, heterocycle, substituted heterocycle, alkyl, substituted alkyl, alkenyl, alkenyl substituted, alkynyl, alkynyl substituted, aralkyl, (CH₂CH₂Y¹³)ᵣ, JAJ, an amino-acid-based moiety, and Y¹²R¹⁰LQR¹¹)_{q}, and each of q and r is an integer value between 1 and 10. By way of example, if the value of q is greater than 1, then Y¹², R¹⁰, L, Q, and R¹¹ can be independent of each other in each repeat unit. In the embodiment described in FIG. 2, Y¹² and Y¹³ is at least one member selected from a group consisting of nitrogen, NH, oxygen, OH, sulfur, SO, SO₂, CO, and carbon.

In several embodiments, Y¹, Y², Y⁴, Y⁶, and X¹ diversity is limited based on the specific chemical identities of the other members of the group Y¹, Y², Y⁴, Y⁶, Y⁸, and X¹ due to restrictions in synthesis. By way of example, if Y¹ is not a CO, then Y²-Y¹¹ is independently selected from Nitrogen, NH, Oxygen, OH, Sulfur, SO, SO₂, CO, or Carbon. As another example, if Y⁴ is not a CO, then Y¹-Y³ and Y⁵-Y¹¹ is independently selected from Nitrogen, NH, Oxygen, OH, Sulfur, SO, SO₂, CO, or Carbon. As yet another example, if Y² is not a NH, then Y¹ and Y²-Y¹¹ is independently selected from Nitrogen, NH, Oxygen, OH, Sulfur, SO, SO₂, CO, or Carbon. As yet another example, if Y⁶ is a ketone, then Y¹-Y⁵ and Y⁷-Y¹¹ is independently selected from Nitrogen, NH, Oxygen, OH, Sulfur, SO, SO₂, CO, or Carbon. As yet another example, if X¹ is not a NH or R³ is a is at least one member selected from a group consisting of CF₃, CHF₂, CH₂F, and other fluoroalkyl groups, then Y¹-Y¹¹ is independently selected from Nitrogen, NH, Oxygen, OH, Sulfur, SO, SO₂, CO, or Carbon.

In several embodiments, R¹-R⁹ is absent or at least one member selected from a group consisting of X¹, hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, halo, a heteroatom, and an amino-acid-based moiety.

In several embodiments, R¹⁰ is absent or at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, (CH₂CH₂Y¹³)ᵣ, JAJ, and an amino-acid-based moiety, wherein A is at least one member selected from a group consisting of C═=O, C═=S, SO, and SO₂, and wherein J is absent or at least one member selected from a group consisting of oxygen, sulfur, NH, and N-alkyl.

In several embodiments, R¹¹ is absent or at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group,) (CH₂CH₂Y¹³r, JAJ, R¹²JAJ-alkyl-, R¹⁵J-alkyl-, (R¹²O)P(═=O)O-alkyl-JAJJ AJ-alkyl-, R¹² JAJ-alkyl-JAJJAJ-alkyl-, JAJheterocyclylMJAJ-alkyl-, (R¹²O)(R¹³O)P(═=O)O-alkyl-, (R¹⁴)₂N— alkyl-, (R¹⁴)₃N+- alkyl-, heterocyclylJ-, carbocyclylJ-, R¹⁵SO₂alkyl-, and R¹⁵SO₂NH.

In several embodiments, R¹² and R¹³ is at least one member selected from a group consisting of hydrogen, metal cation, aryl, carbocycle, substituted aryl, substituted carbocycle, heterocycle, substituted heterocycle, alkyl, substituted alkyl, alkenyl, substituted alkenyl, alkynyl, substituted alkynyl, and aralkyl; or R¹² and R¹³ together are alkyl, substituted alkyl, aralkyl, thereby forming a ring.

In several embodiments, R¹⁴ is at least one member selected from hydrogen or alkyl; R¹⁵ is at least one member selected from a group consisting of H, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, and an amino acid side chain; M is absent or is alkyl; and Z¹-Z³ is absent or independently selected from hydrogen and fluorine.

In several embodiments, R¹⁰ and R¹¹ together are alkyl-A-alkyl, alkyl-JAJ-alkyl, JAJ-alkyl-JAJ-alkyl, JAJ-alkyl-JAJ, or alkyl-A, substituted alkyl, aralkyl, thereby forming a ring; L is at least one member selected from a group consisting of C═=O, C═=S, SO, and SO₂; and Q is absent or at least one member selected from a group consisting of carbon, oxygen, NH, and N-alkyl.

In several embodiments, X² is absent or at least one member selected from a group consisting of hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, (CH₂CH₂Y¹³)ᵣ, JAJ, and an amino-acid-based moiety.

By way of example, the term “amino-acid-based moiety” shall be given its ordinary meaning and shall also refer to both standard and non-standard, including derivatives and analogs, halo and other heteroatoms. The term also refers to side chain or group coming off the amino acid unit, typically alpha to the carboxyl group. Further still, in relevant instances, the term also includes a single or series of bonded amino acid and/or amino alcohols with previously states groups substituted on said chain, including a combination of those groups.

The variables that are represented are independent of each other and may be enantiomers, stereoisomeric forms, mixtures of enantiomers, diastereomers, mixtures of diastereomers, prodrugs, hydrates, solvates, and racemates of the above mentioned compounds and pharmaceutically acceptable salts thereof.

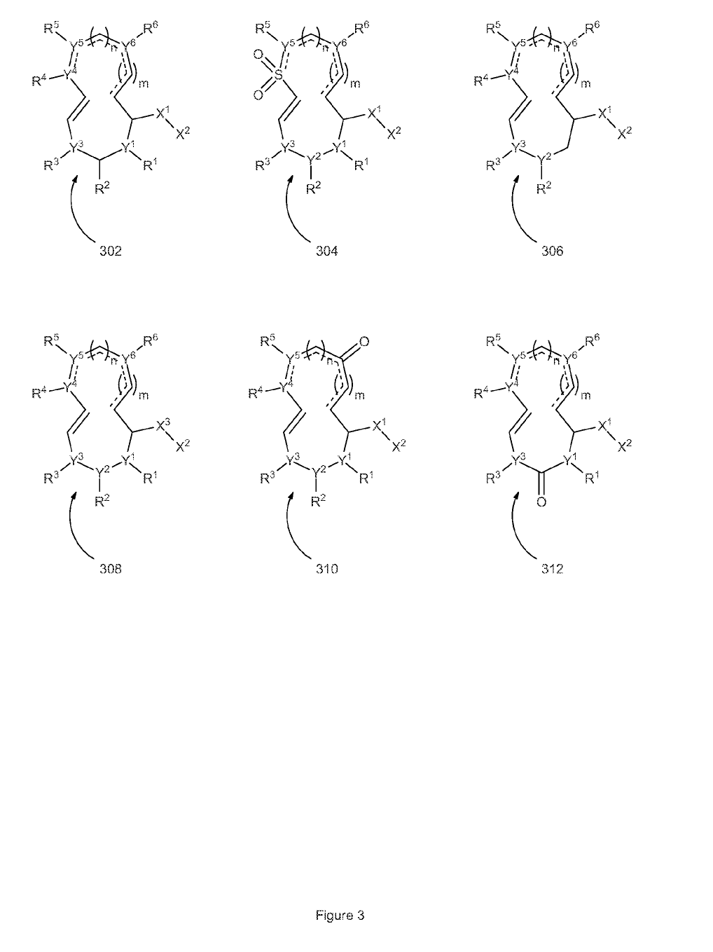

In additional embodiments, a proteasome inhibiting core ring structure is at least one structure selected from Formulas I to III as shown in FIG. 3. In certain such embodiments, Y¹ to is at least one member selected from a group consisting of oxygen, sulfur, SO, SO₂, CO, and carbon and each of n and m is an integer value equal to 0, 1, or 2. In the embodiment shown in FIG. 3 (which is a non-limiting example), n and m may both equal 1. In certain embodiment of the present invention, however, if n equals 0, then m equals 1. In certain other embodiments of the present invention, if n equals 1, then m equals 2.

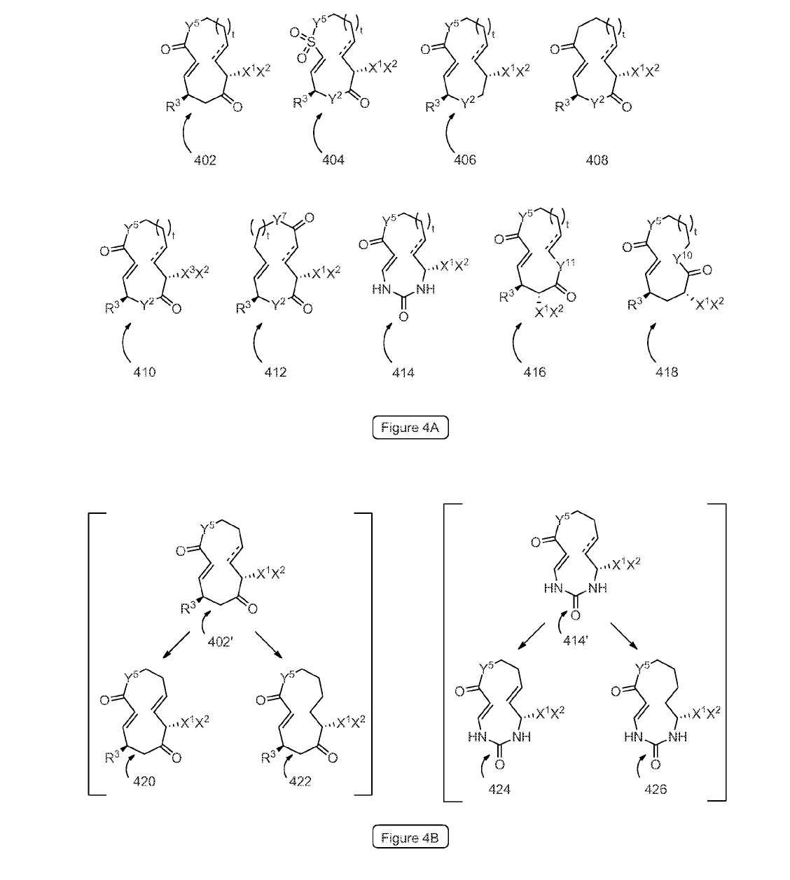

In several embodiments, a core ring structure is at least one structure selected from Formulas 1 to 111 as shown in FIG. 4A, wherein each of Y², Y⁵, Y⁷, Y¹⁰, and Y¹¹ is at least one member selected from a group consisting of nitrogen, NH, oxygen, OH, sulfur, SO, SO₂, CO, and carbon; wherein X³ is one member selected from a group consisting of oxygen, sulfur, SO, SO₂, CO, and carbon; and, CH₂O, COH, CO₂H, halide, P(X²)₃, BOH, B(OH)₂, aryl, carbocycle, substituted aryl, substituted carbocycle, heterocycle, substituted heterocycle, alkyl, substituted alkyl, alkenyl, alkenyl substituted, alkynyl, alkynyl substituted, aralkyl, (CH₂CH₂Y¹³)ᵣ, JAJ, an amino-acid-based moiety, and (Y¹²R¹⁰LQR¹¹)_{q}, and each of q and r is an integer value between 1 and 10; and wherein t is an integer value between 0 and 2.

Structures 402-416 are non-limiting examples of structural derivations and analogs of inventive proteasome inhibitor family including newly developed urea containing core moiety. Those skilled in the art will understand the nomenclature concepts. For facilitating discussion, certain non-limiting examples are shown and discussed below.

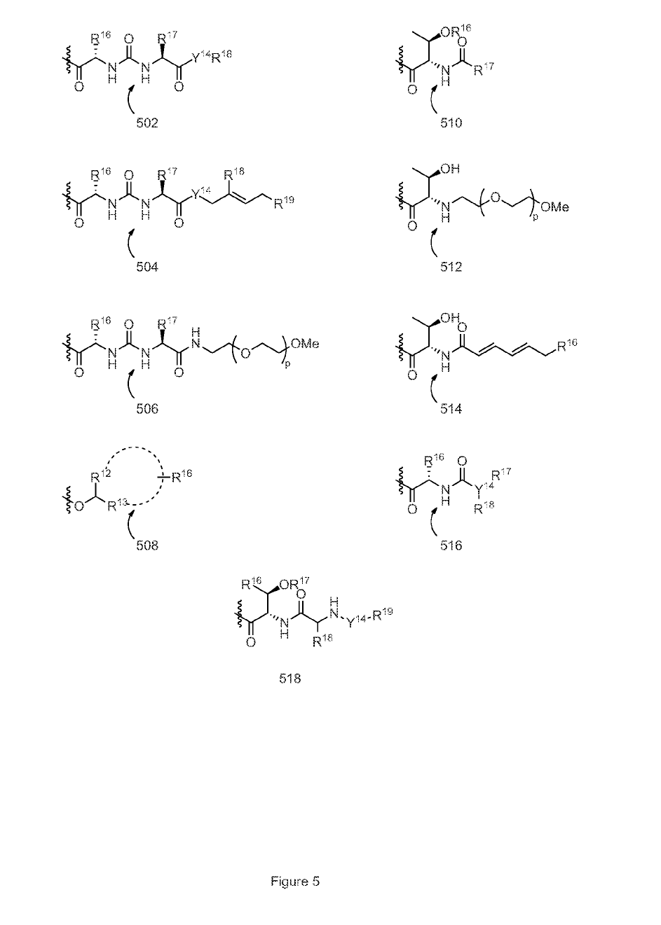

In several embodiments, X¹ or X² comprise the structure shown in FIG. 5. In this embodiment, Y¹⁴ is at least one member selected from a group consisting of nitrogen, NH, oxygen, OH, sulfur, SO, SO₂, CO, and carbon, and R¹² and R¹³ is at least one member selected from a group consisting of hydrogen, metal cation, aryl, carbocycle, substituted aryl, substituted carbocycle, heterocycle, substituted heterocycle, alkyl, substituted alkyl, alkenyl, substituted alkenyl, alkynyl, substituted alkynyl, and aralkyl. In some embodiments, R¹² and R¹³ form a ring that is at least one member selected from a group consisting of alkyl, substituted alkyl, and aralkyl. In certain embodiment, R¹⁴ is at least one member selected from a group consisting of hydrogen and alkyl, and R¹⁵ is at least one member selected from a group consisting of H, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, and an amino-acid-based moiety.

In several embodiments, each of R¹⁶, R¹⁷, R¹⁸ and R¹⁹ is absent or at least one member selected from a group consisting of X¹, hydrogen, CF₃, aryl, carbocycle, substituted aryl, substituted carbocycle, heterocycle, substituted heterocycle, alkyl, substituted alkyl, alkenyl, substituted alkenyl, alkynyl, substituted alkynyl, aralkyl, carboxyl, aminocarbonyl, alkylsulfonylaminocarboxyl, alkoxycarbonyl, heteroaralkyl, an N-terminal protecting group, an O-terminal protecting group, halo, a heteroatom, and an amino-acid-based moiety. In one embodiment of the present invention, p is an integer value between 1 and 20.

Several embodiments disclosed herein, among other things, provides compounds comprising of novel proteasome inhibitors core formulas (e.g., 302, 304, 306, 308, 310, and 312) that represent preferred embodiments of the present invention and also provide novel schemes of synthesis for proteasome inhibitors.

The reaction schemes provided depict basic core derivatives and potential structural analogs in simplified terms with simplified reagents/reactants. Most are available through a commercial source while others need to be synthesized (which is within the ordinary skill in the art based on the disclosure herein). For those skilled in the art, simplified terms such as peptide coupling, cross-metathesis or olefin metathesis, Redox (reduction-oxidation) reactions, and other coupling named reactions are stated. Peptide coupling includes, but is not limited to, Dicyclohexylcarbodiimide (DCC), diisopropylcarbodiimide (DIC), 1-hydroxy-7-aza-benzotriazole (HOAt), 1-hydroxybenzotriazole (HOBt), Ethyl (hydroxyimino)cyanoacetate (Oxyma), N-(3-Dimethylaminopropyl)-N′-ethylcarbodiimide (EDC), 4-(N,Ndimethylamino)pyridine (DMAP), (Benzotriazol-1-yloxy)tris(dimethylamino)phosphonium hexafluorophosphate (BOP), (O-(7-azabenzotriazol-1-yl)-N,N,N′,N′-tetramethyluronium hexafluorophosphate) (HATU), 0-(Benzotriazol-1-yl)-N,N,N′,N′-tetramethyluronium hexafluorophosphate (HBTU), 3-(Diethylphosphoryloxy)-1,2,3-benzotriazin-4(3H)-one (DEPBT), 2-Isobutoxy-1-isobutoxycarbonyl-1,2-dihydroquinoline (IIDQ), 2-Ethoxy-1-ethoxycarbonyl-1,2-dihydroquinoline (EEDQ), and CarbonyldiImidazole (CDI). These are useful for forming, for example, amides, esters and thioesters.

Cross-Metathesis or olefin metathesis includes, but is not limited to, Grubbs catalysts, Hoveyda-Grubbs catalysts, Schrock catalysts, and other organometallic compounds. Redox (reduction-oxidation) reactions my include, but are not limited to, Ozone, nitrate compounds, Hydrogen peroxide and other inorganic peroxides, Sulfuric acid, Persulfuric acids, halogen compounds, Hypochlorite and other hypohalite compounds, Hexavalent chromium compounds, Permanganate compounds, Silver oxide, Osmium tetroxide, 2,2′-Dipyridyldisulfide, Lithium aluminum hydride, Sodium amalgam, Sodium borohydride, Compounds containing the Sn²⁺ ion, Compounds containing the Fe²⁺ ion, Hydrazine, Diisobutylaluminum hydride, Lindlar catalyst, Oxalic acid, Formic acid, Phosphites, hypophosphites, phosphorous acid, Dithiothreitol, Electropositive elemental metals, also including named reactions such as Dess-Martin oxidation, Swern reduction, Mitsunobu Reaction, Meerwein-Ponndorf-Verley Reduction among others. Other coupling reaction/named reactions my include, but are not limited to, Horner-Wadsworth-Emmons reaction, Wittig reaction, Fukuyama coupling, Negishi coupling, Heck coupling, Buchwald-Hartwig reaction, Grignard reaction, palladium, cobalt, nickel among others.

Protecting groups include, but are not limited to, Acetyl (Ac), Benzyl (Bn), β-Methoxyethoxymethyl ether (MEM), Pivaloyl (Piv), Silyl ether, Carbobenzyloxy (Cbz), tert-Butyloxycarbonyl (BOC), 9-Fluorenylmethyloxycarbonyl (FMOC), Acetyl (Ac), Benzoyl (Bz), p-Methoxybenzyl (PMB), Carbamate group, Tosyl (Ts), tert-Butyldimethylsilyl chloride (TBDMSCl), Trimethylsilyl chloride, Acetals and Ketals, Acylals, Dithianes, Methyl, Benzyl, tert-Butyl, and propargyl alcohols.

Deprotecting groups include, but are not limited to Acid, base, hydrogenolysis, fluoride ion and other halogenated derivatives, heating, metal salts, oxidizing agents, reducing agents, organometallic, Favorskii reaction, and Corey-Winter Olefination.

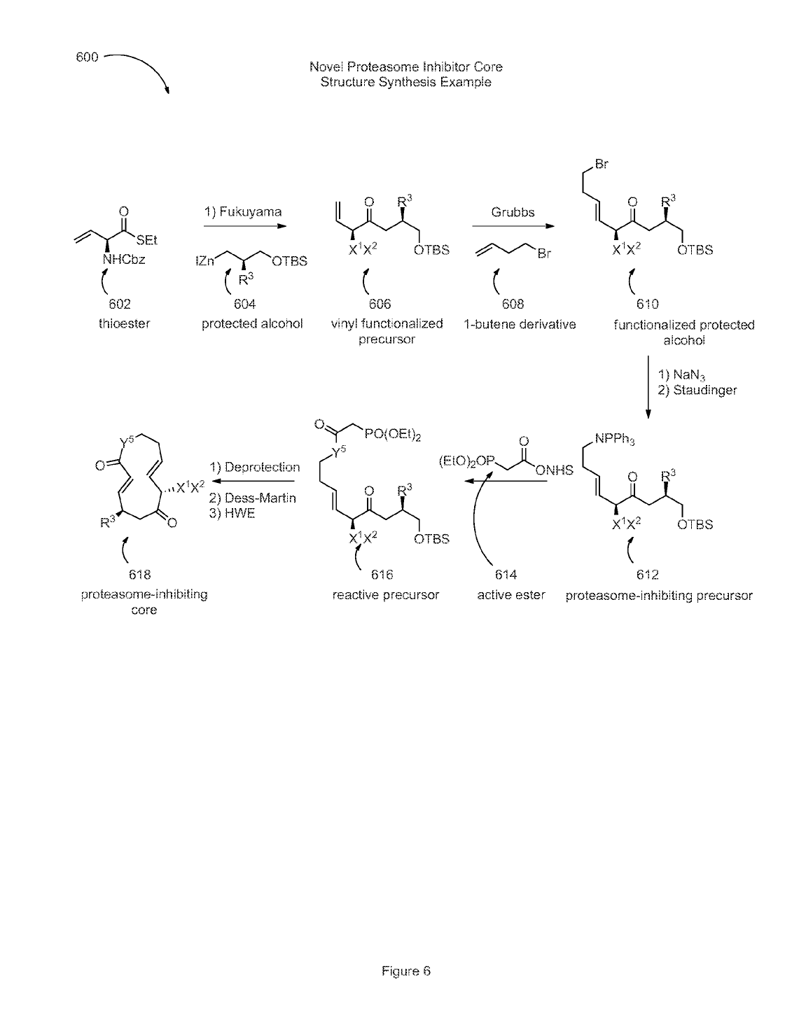

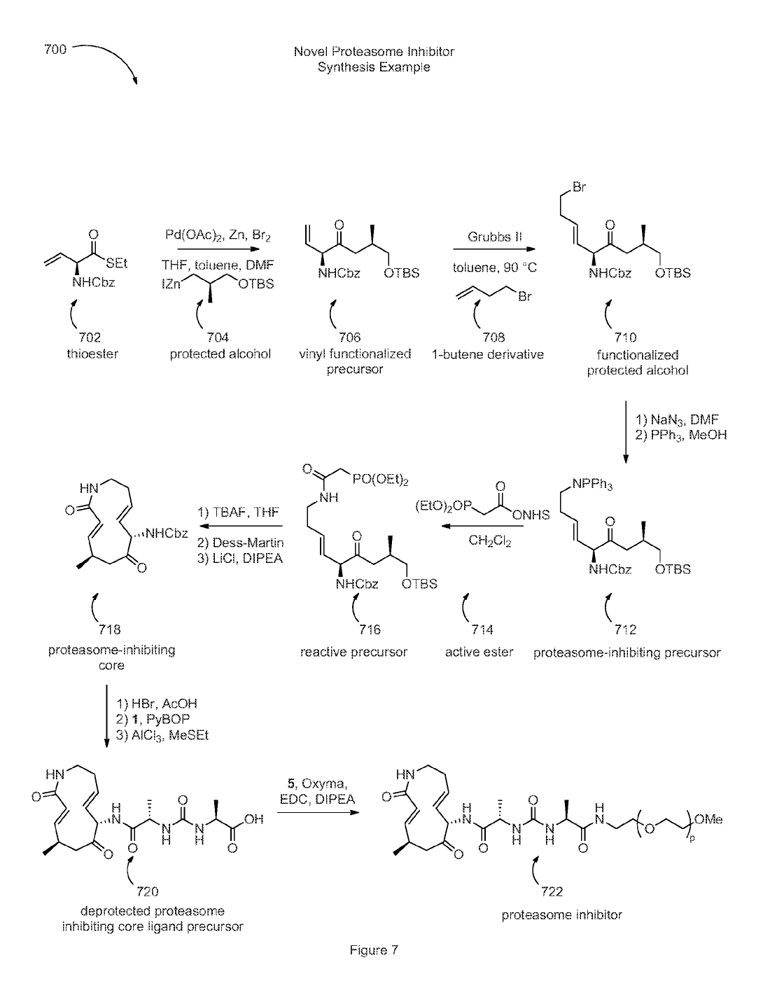

Schemes 600/700, in accordance with several embodiments of the present invention, depict synthesis of one embodiment of a proteasome inhibitor core structure and proteasome inhibitor, as shown in FIGS. 6 & 7, respectively. These schemes depict use of a functionalized thioester 602/702, which can be coupled together with protected alcohol 604/704 to provide vinyl functionalized precursor 606/706 under Fukuyama conditions. A cross-metathesis reaction with a 1-butene derivative (608/608) and an olefin metathesis catalyst, such as Grubbs catalyst, provides a functionalized protected alcohol 610/610. By performing a nucleophilic substitution on the halide of 610/710 with azide, followed by a Staudinger reaction, a proteasome inhibiting precursor 612/712 is prepared. This is coupled with the phosphonoacetic acid active ester 614/714, which provides a precursor to a Horner-Wadsworth-Emmons reaction (reactive precursor 616/716). After deprotection of the alcohol, this then can be oxidized to the aldehyde using oxidizing conditions such as Des s-Martin conditions, followed by a HWE cyclization to create proteasome inhibiting core 618/718. Furthermore, scheme 700 describes one of the possible ligand couplings to said proteasome inhibiting core 718. Scheme 700 continues with the deprotection of the proteasome inhibiting core 718 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 720. Then coupling on compound 5 is performed to produce the final proteasome inhibiting core with ligand 722.

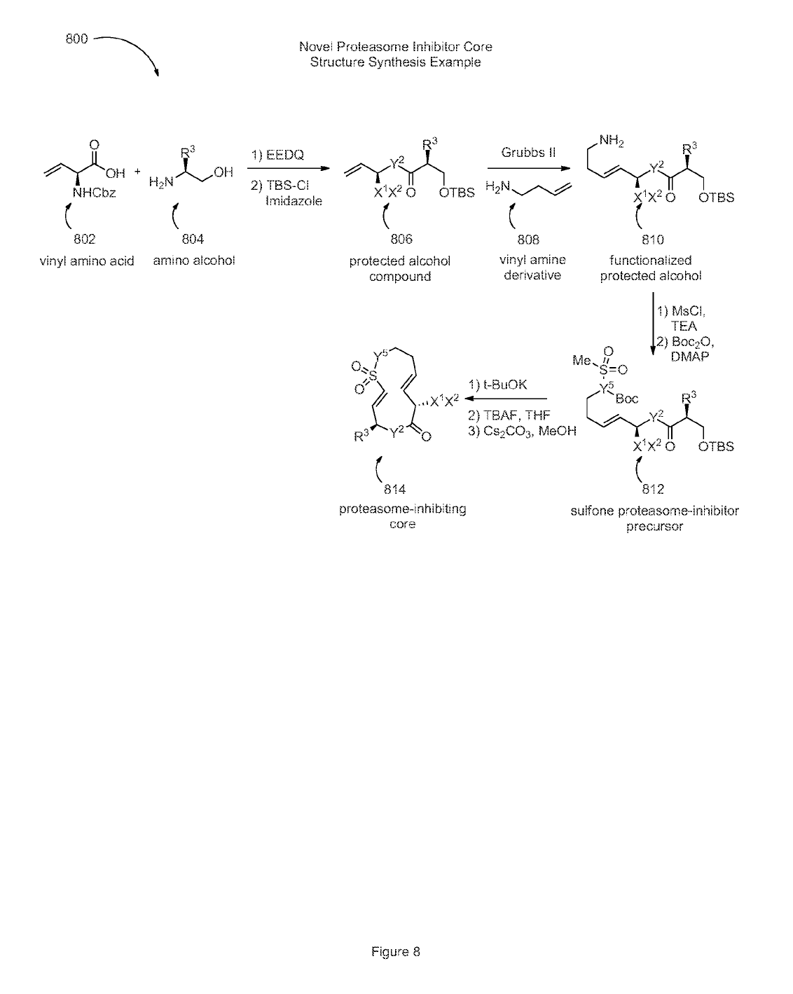

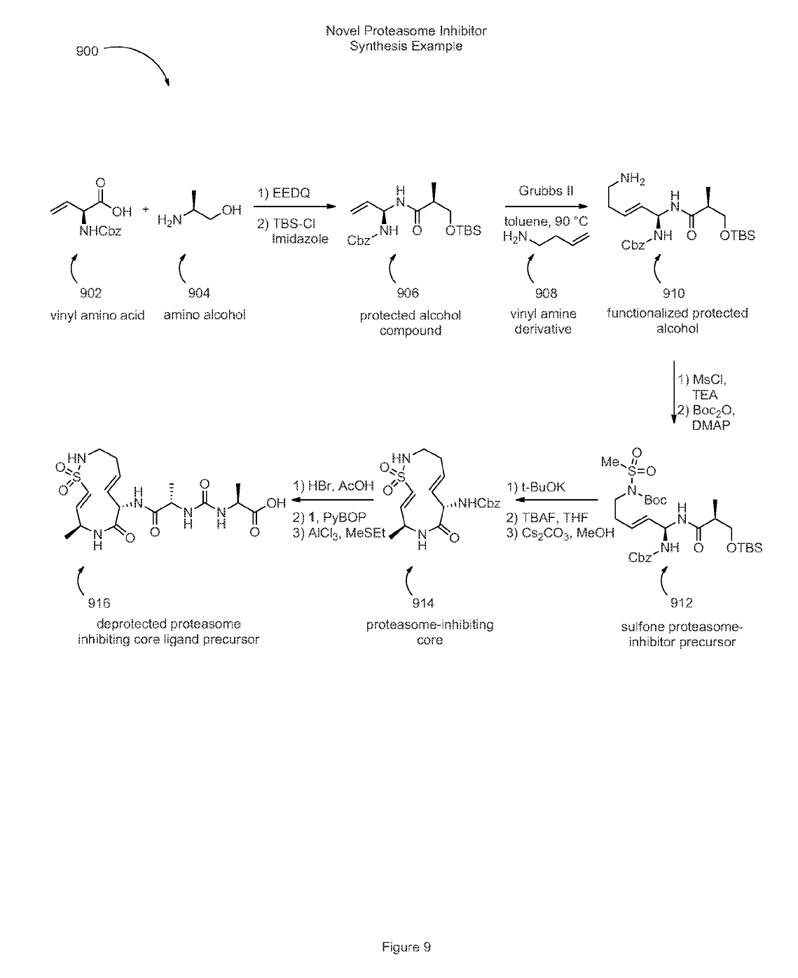

Schemes 800/900, in accordance with several embodiments of the present invention, depict synthesis of an additional proteasome inhibitor core structure and proteasome inhibiting core-ligand precursor, as shown in FIGS. 8 & 9, respectively. These schemes include using a vinyl amino acid 802/902 and an amino alcohol 804/904, combined by a peptide coupling reaction, then an alcohol protection, which produces a protected alcohol compound 806/906. A cross-metathesis reaction with a vinyl amine derivative (808/908) and an olefin metathesis catalyst, such as Grubbs catalyst, provides a functionalized protected alcohol 810/910. The primary amine is subsequently treated with methanesulfonyl chloride and triethylamine and then protected with tert-Butyl carbamate and DMAP to provide sulfone proteasome-inhibitor precursor 812/912. Upon addition of a strong base followed by deprotection of the primary alcohol, and finally a reducing agent such as caesium carbonate, a proteasome inhibiting core 814/914 is produced. Furthermore, scheme 900 describes one of the possible ligand couplings to said proteasome inhibiting core 914, continuing with the deprotection of the proteasome inhibiting core 914 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 916. Further steps may be performed to provide a desired ligand on said proteasome inhibiting core.

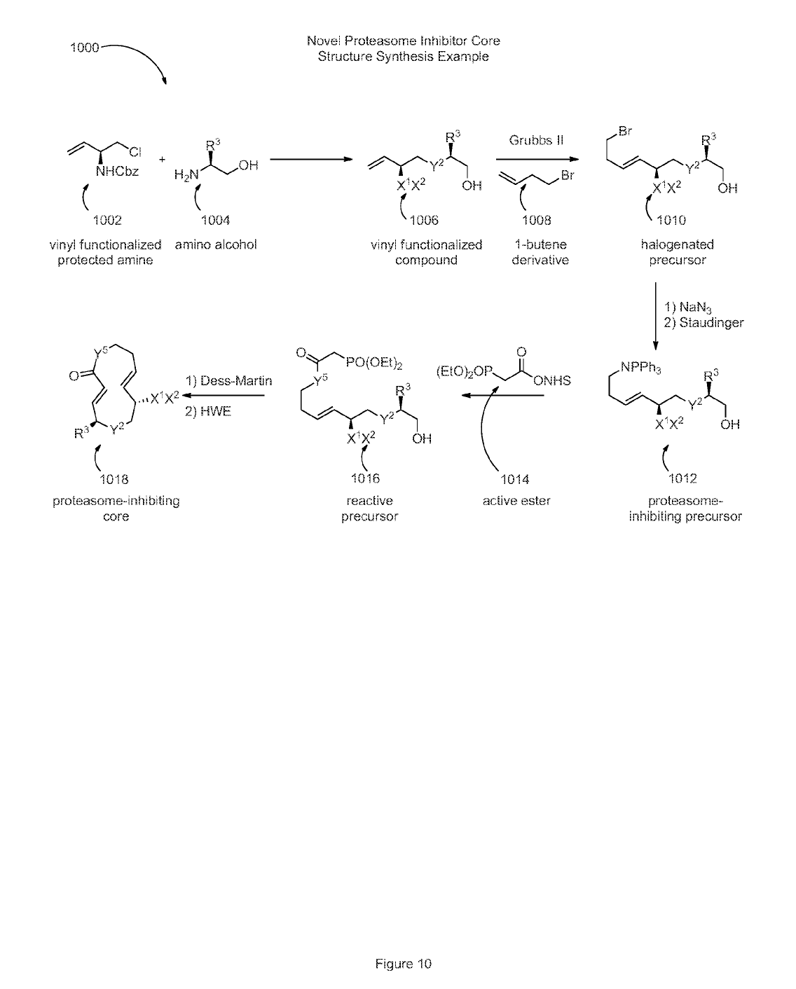

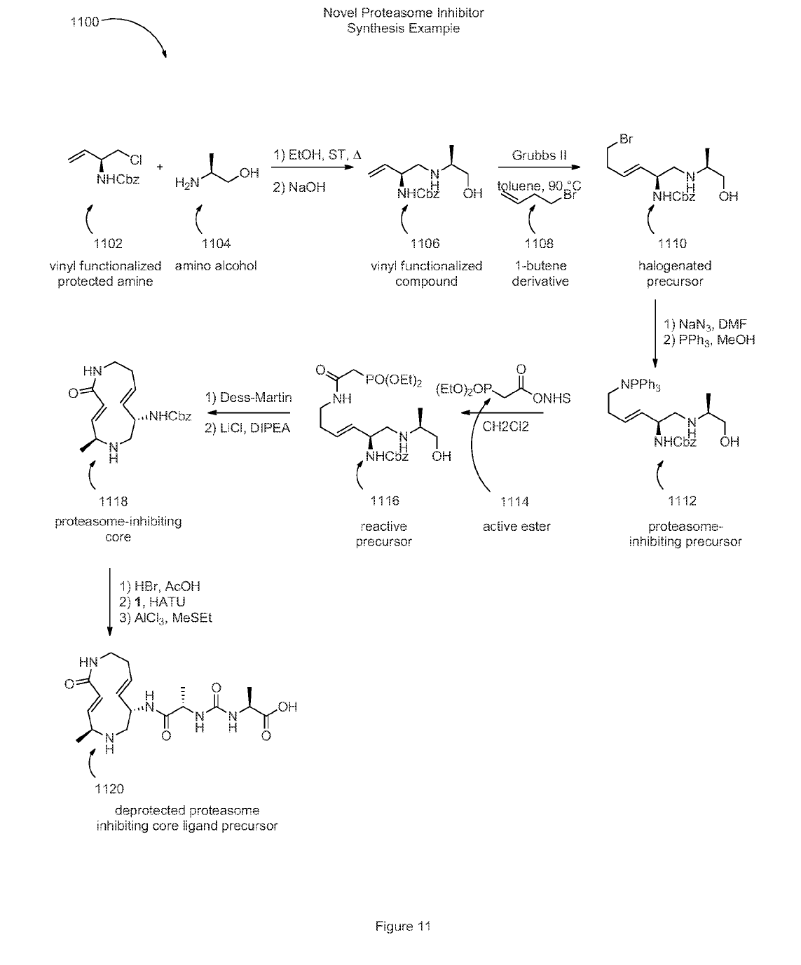

Schemes 1000/1100, in accordance with several embodiments of the present invention, depict an example of an additional proteasome inhibitor core structure and proteasome inhibiting core-ligand precursor, as shown in FIGS. 10 & 11, respectively. These schemes include using a vinyl functionalized protected amine 1002/1102 and an amino alcohol 1004/1104, combined by a nucleophilic substitution reaction, which produces vinyl functionalized compound 1006/1106. A cross-metathesis reaction with a 1-butene derivative (1008/1108) and an olefin metathesis catalyst, such as Grubbs catalyst, provides a halogenated precursor 1010/1110. By performing a nucleophilic substitution of the halide with azide followed by the Staudinger reaction prepares proteasome inhibiting precursor 1012/1112. This is coupled with the phosphonoacetic acid active ester 1014/1114, which provides the precursor to the Horner-Wadsworth-Emmons reaction (reactive precursor 1016/1116). This then can be oxidized to the aldehyde using oxidizing conditions, such as Dess-Martin conditions, followed by a HWE cyclization to create proteasome inhibiting core 1018/1118. Furthermore scheme 1100 describes one of the possible ligand couplings to said proteasome inhibiting core 1118, continuing with the deprotection of the proteasome inhibiting core 1118 to provide a free amine proteasome inhibiting core, which is coupled with compound 1 to provide a proteasome inhibiting core ligand precursor. Removal of the protecting group on proteasome inhibiting core ligand precursor provides deprotected proteasome inhibiting core ligand precursor 1120. Further steps may be performed to provide desired ligand on said proteasome inhibiting core.

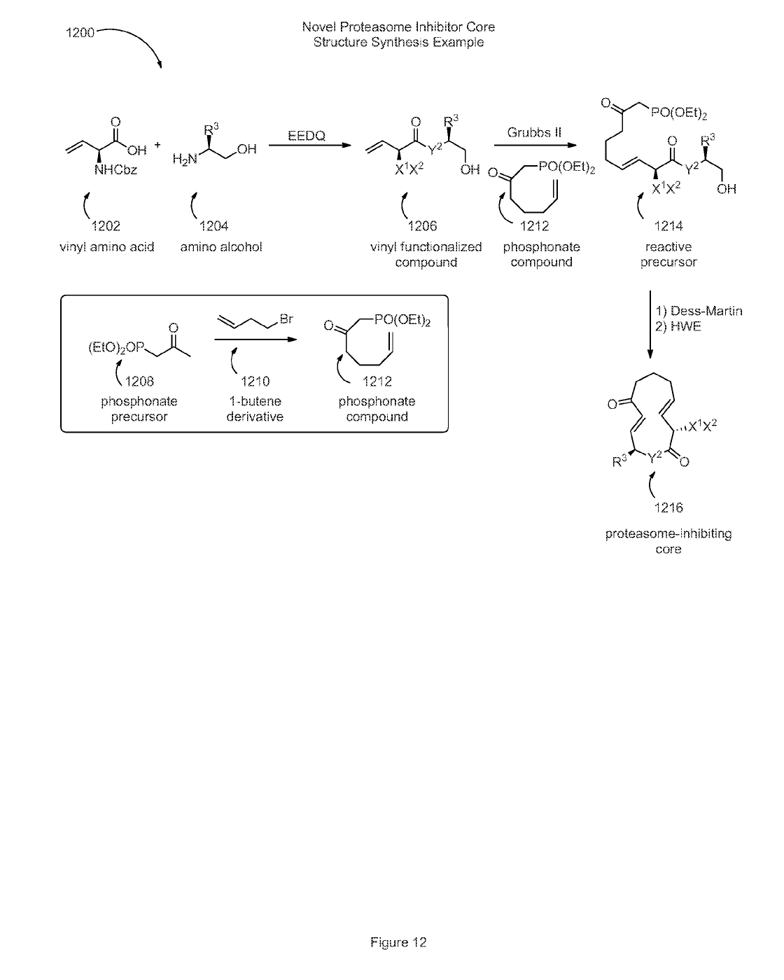

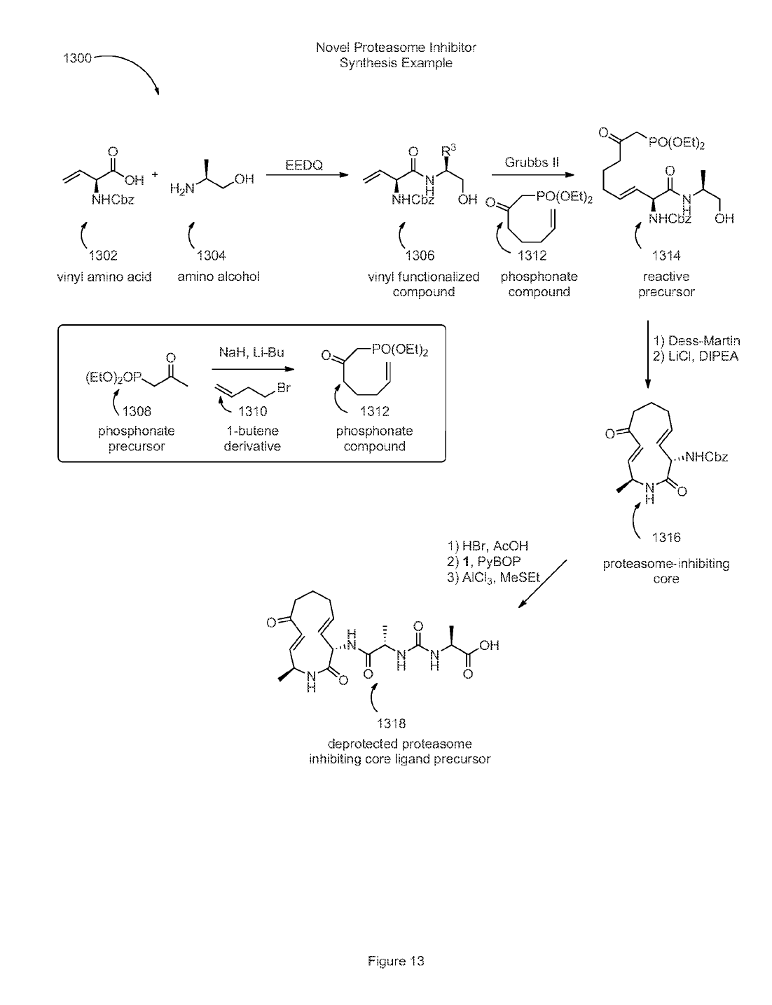

Schemes 1200/1300, in accordance with several embodiments of the present invention, depict synthesis of an additional proteasome inhibitor core structure and proteasome inhibiting core-ligand precursor, as shown in FIGS. 12 & 13, respectively. These schemes include using a vinyl amino acid 1202/1302 and an amino alcohol 1204/1304, combined by a peptide coupling reaction, which produces vinyl functionalized compound 1206/1306. A cross-metathesis reaction with phosphonate compound 1212/1312 (Synthesized by a substitution reaction involving phosphonate precursor 1208/1308 and a 1-butene derivative 1210/1310) and an olefin metathesis catalyst, such as Grubbs catalyst, provides reactive precursor 1214/1314. This then can be oxidized to the aldehyde using oxidizing conditions such as Dess-Martin conditions, followed by a HWE cyclization, to create proteasome inhibiting core 1216/1316. Furthermore scheme 1300 describes one of the possible ligand couplings to said proteasome inhibiting core 1316 continuing with the deprotection of the proteasome inhibiting core 1316 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 1318. Further steps may be performed to provide desired ligand on said proteasome inhibiting core.

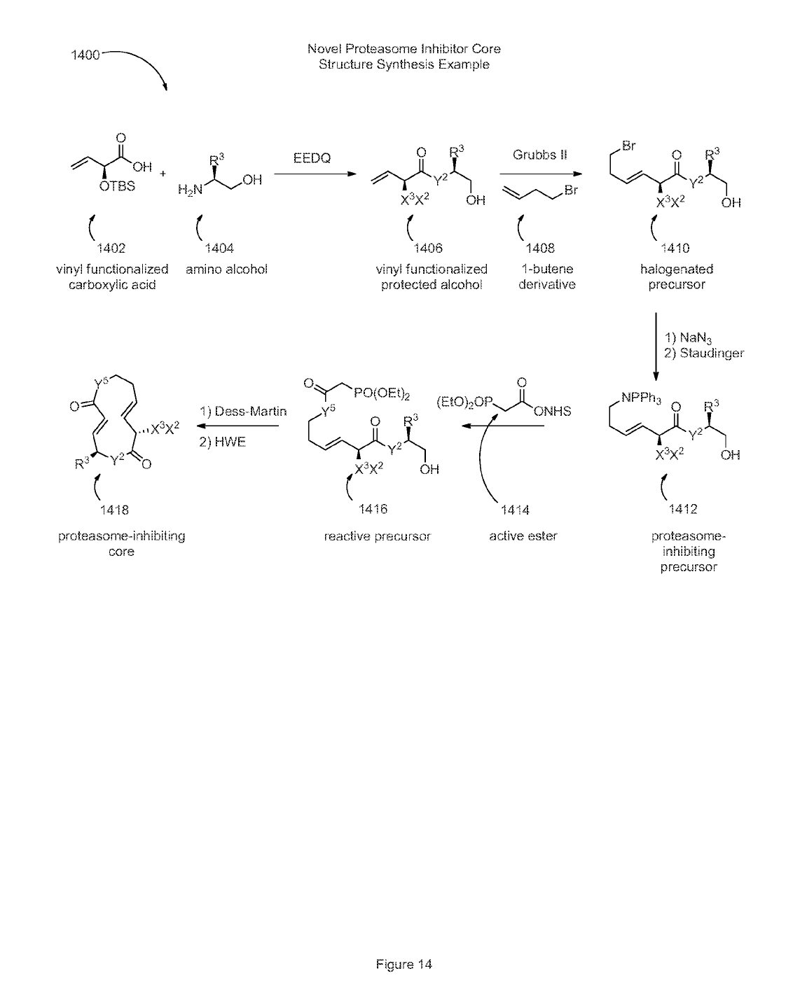

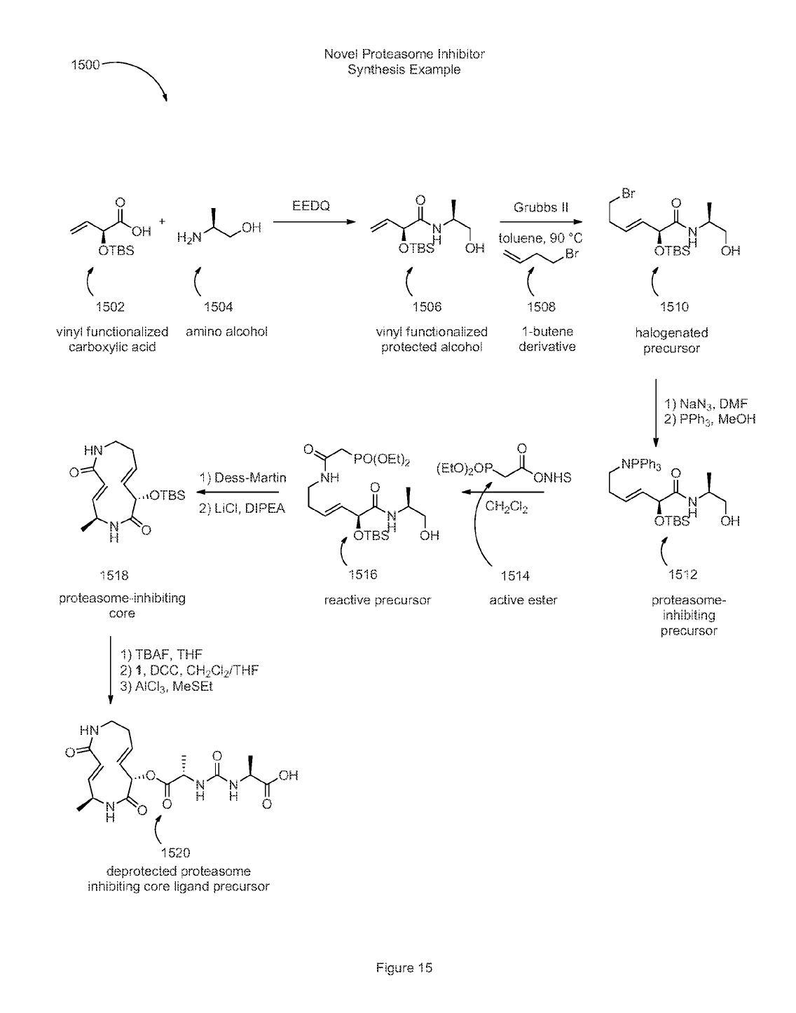

Schemes 1400/1500, in accordance with several embodiments of the present invention, depict synthesis of an additional proteasome inhibitor core structure and proteasome inhibiting core-ligand precursor, as shown in FIGS. 14 & 15, respectively. These schemes include using a vinyl functionalized carboxylic acid 1402/1502 and an amino alcohol 1404/1504, combined by a peptide coupling reaction, which produces vinyl functionalized protected alcohol 1406/1506. A cross-metathesis reaction with a 1-butene derivative (1408/1508) and an olefin metathesis catalyst, such as Grubbs catalyst, provides halogenated precursor 1410/1510. By performing a nucleophilic substitution of the halide with azide followed by the Staudinger reaction, proteasome inhibiting precursor 1412/1512 is prepared. This is coupled with the phosphonoacetic acid active ester 1414/1514, which provides the precursor to the Horner-Wadsworth-Emmons reaction (reactive precursor 1416/1516). This then can be oxidized to the aldehyde using oxidizing conditions such as Des s-Martin conditions, followed by a HWE cyclization, to create proteasome inhibiting core 1418/1518. Furthermore scheme 1500 describes one of the possible ligand couplings to said proteasome inhibiting core 1518 continuing with the deprotection of the proteasome inhibiting core 1518 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 1520. Further steps may be performed to provide desired ligand on said proteasome inhibiting core.

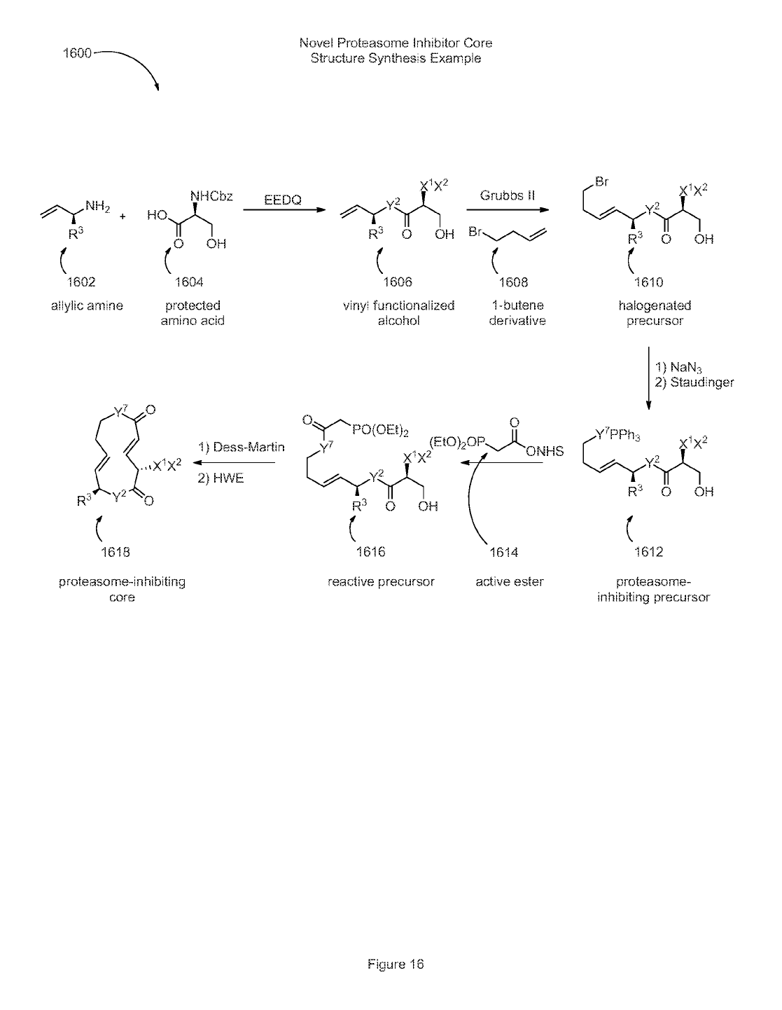

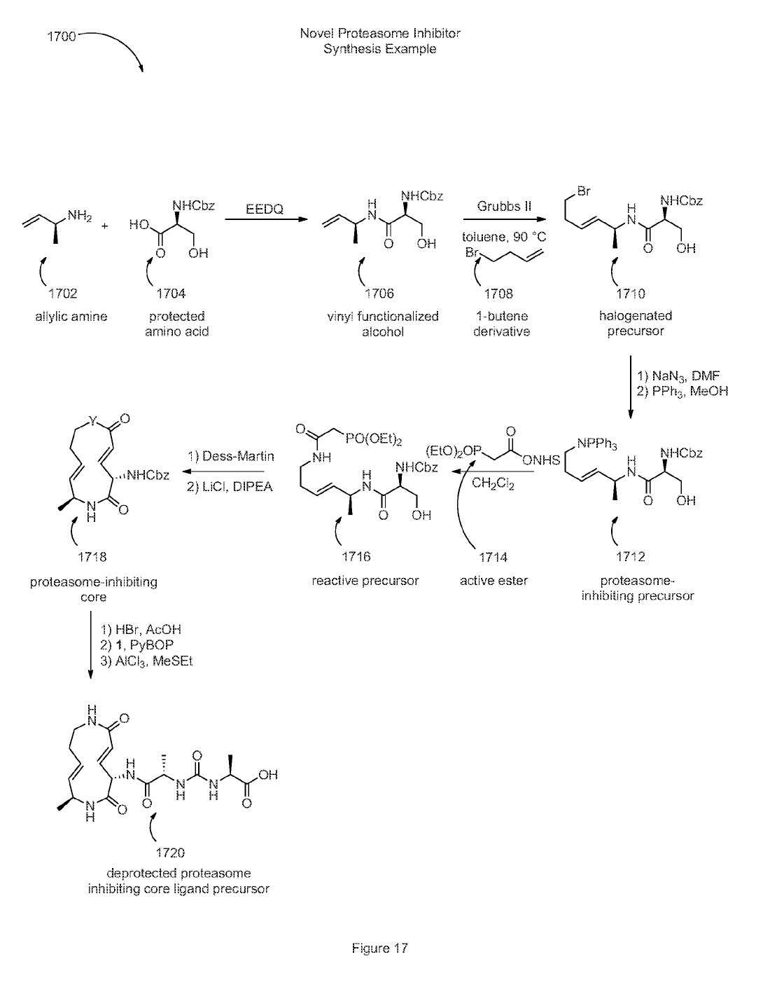

Schemes 1600/1700, in accordance with several embodiments of the present invention, depict synthesis of an additional proteasome inhibitor core structure and proteasome inhibiting core-ligand precursor, as shown in FIGS. 16& 17, respectively. These schemes include using an allylic amine 1602/1702 and a protected amino acid 1604/1704 combined by a peptide coupling reaction to vinyl functionalized alcohol 1606/1706. A cross-metathesis reaction with a 1-butene derivative (1608/1708) and an olefin metathesis catalyst, such as Grubbs catalyst, provides halogenated precursor 1610/1710. To introduce the last nitrogen a nucleophilic substitution is performed on the halide with azide followed by the Staudinger reaction to prepare proteasome inhibiting precursor 1612/1712. This is coupled with the phosphonoacetic acid active ester 1614/1714, which provides the precursor to the Horner-Wadsworth-Emmons reaction (reactive precursor 1616/1716). This then can be oxidized to the aldehyde using oxidizing conditions such as Des s-Martin conditions followed by a HWE cyclization to create proteasome inhibiting core 1618/1718. Furthermore scheme 1700 describes one of the possible ligand couplings to said proteasome inhibiting core 1718, continuing with the deprotection of the proteasome inhibiting core 1718 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 1720. Further steps may be performed to provide desired ligand on said proteasome inhibiting core.

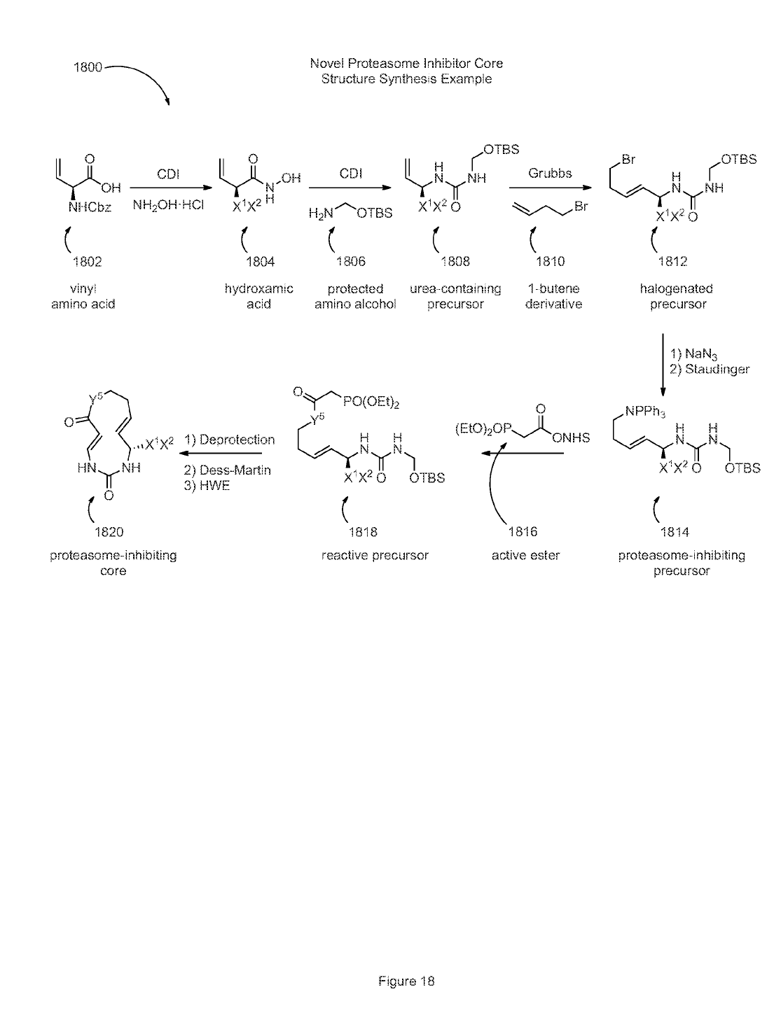

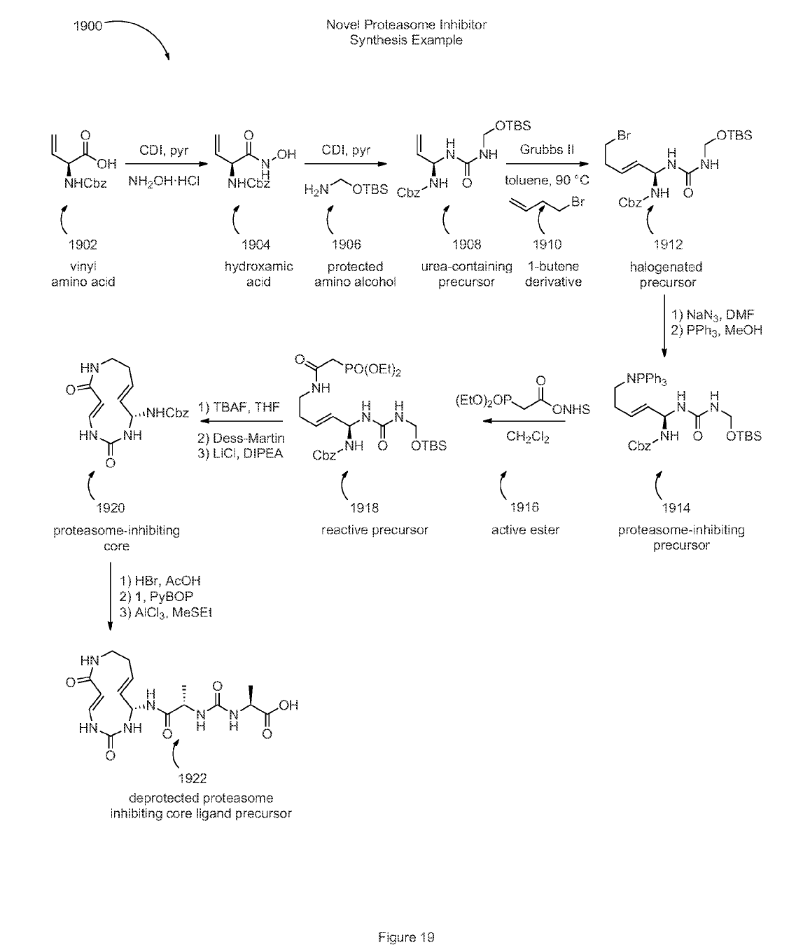

Schemes 1800/1900, in accordance with several embodiments of the present invention, depict synthesis of an additional proteasome inhibitor core structure and proteasome inhibiting core-ligand precursor, as shown in FIGS. 18 & 19, respectively. These schemes include using a vinyl amino acid 1802/1902, Carbonyldiimidazole, and Hydroxylamine hydrochloride to synthesize hydroxamic acid 1804/1904 which can be retreated with Carbonyldiimidazole and a protected amino alcohol 1806/1906, to synthesize the urea containing precursor 1808/1908. A cross-metathesis reaction with a 1-butene derivative (1810/1910) and an olefin metathesis catalyst, such as Grubbs catalyst, provides halogenated precursor 1812/1912. To introduce the last nitrogen, a nucleophilic substitution is performed on the halide with azide followed by the Staudinger reaction to prepare proteasome inhibiting precursor 1814/1914. This is coupled with the phosphonoacetic acid active ester 1816/1916, which provides the precursor to the Horner-Wadsworth-Emmons reaction (reactive precursor 1818/1918). After deprotection of the alcohol, this then can be oxidized to the aldehyde using oxidizing conditions such as Dess-Martin conditions, followed by a HWE cyclization to create proteasome inhibiting core 1820/1920. Furthermore scheme 1900 describes one of the possible ligand couplings to said proteasome inhibiting core 1920, continuing with the deprotection of the proteasome inhibiting core 1920 to provide a free amine proteasome inhibiting core which is coupled with compound 1 to provide proteasome inhibiting core ligand precursor. Removal of the protecting group on proteasome inhibiting core ligand precursor provides deprotected proteasome inhibiting core ligand precursor 1922. Further steps may be performed to provide a desired ligand on the proteasome inhibiting core.

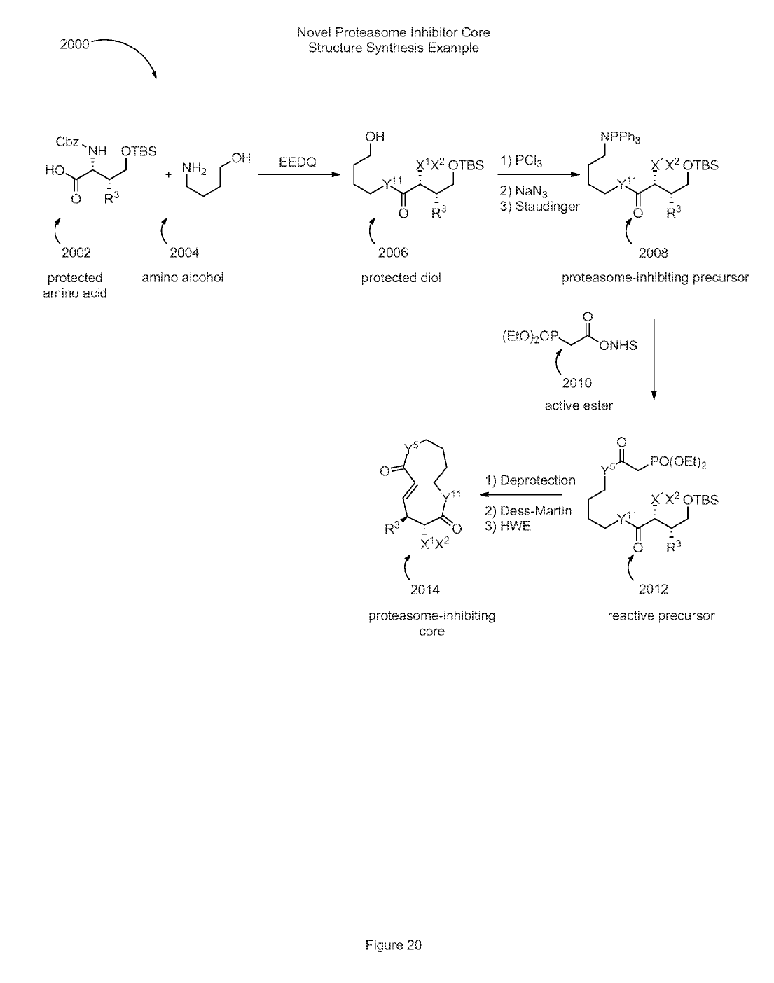

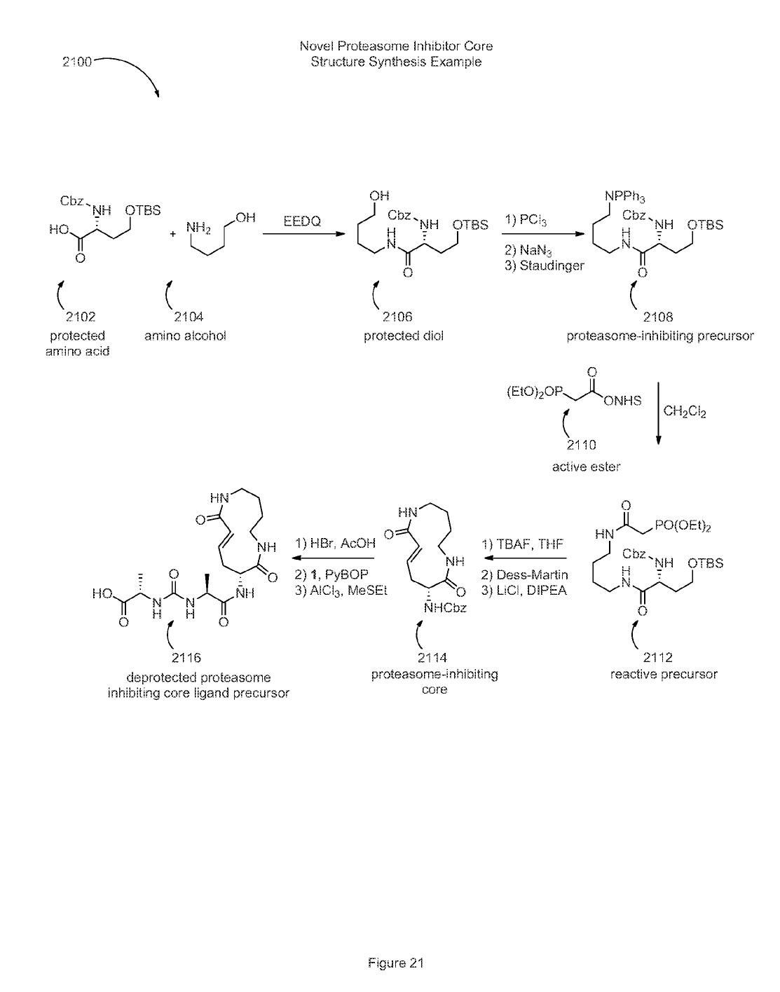

Schemes 2000/2100, in accordance with several embodiments of the present invention, depict synthesis of an additional proteasome inhibitor core structure and proteasome inhibiting core-ligand precursor, as shown in FIGS. 20 & 21, respectively. These schemes include using protected amino acid 2002/2102 and an amino alcohol 2004/2104 combined by a peptide coupling reaction, which produces a protected diol 2006/2106. Treating the free alcohol with halogenation agent, followed with an azide salt, and finally followed by the Staudinger reaction, prepares proteasome inhibiting precursor 2008/2108. This is coupled with the phosphonoacetic acid active ester 2010/2110, which provides the precursor to the Horner-Wadsworth-Emmons reaction (reactive precursor 2012/2112). After deprotection of the alcohol, this then can be oxidized to the aldehyde using oxidizing conditions such as Dess-Martin conditions, followed by a HWE cyclization, to create proteasome inhibiting core 2014/2114. Furthermore scheme 2100 describes one of the possible ligand couplings to proteasome inhibiting core 2114 continuing with the deprotection of the proteasome inhibiting core 2114 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2116. Further steps may be performed to provide desired ligand on said proteasome inhibiting core.

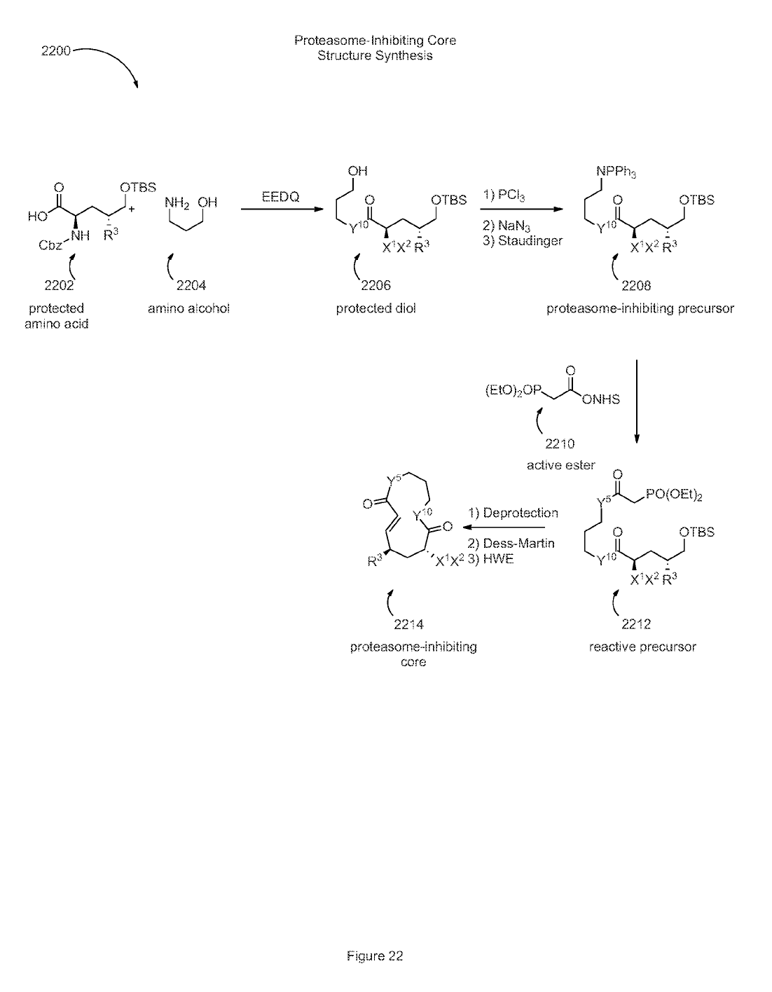

Scheme 2200, in accordance with several embodiments of the present invention, depicts synthesis of additional proteasome inhibitor core structures, as shown in FIG. 22. This scheme includes using protected amino acid 2202 and an amino alcohol 2204 combined by a peptide coupling reaction which produces a protected diol 2206. Treating the free alcohol with halogenation agent, followed with an azide salt, and finally followed by the Staudinger reaction prepares proteasome inhibiting precursor 2208. This is coupled with the phosphonoacetic acid active ester 2210 which provides the precursor to the Horner-Wadsworth-Emmons reaction (reactive precursor 2212). After deprotection of the alcohol this then can be oxidized to the aldehyde using oxidizing conditions such as Dess-Martin conditions followed by a HWE cyclization to create proteasome inhibiting core 2214.

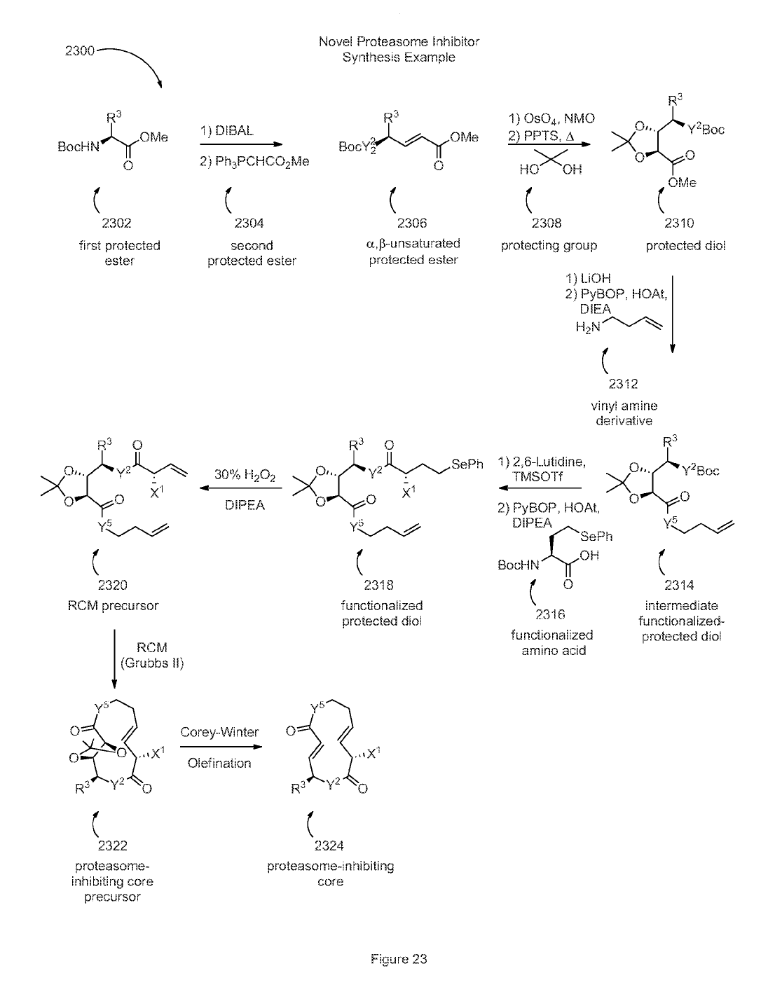

Scheme 2300, in accordance with several embodiments of the present invention, depicts synthesis of additional proteasome inhibitor core structures, as shown in FIG. 23. This scheme includes using of a reduction of a first protected ester 2302 to form an aldehyde. Coupling said aldehyde with a second protected ester 2304, which is different from said first protected ester, to form an α,β-unsaturated protected ester 2306. Oxidizing said α,β-unsaturated protected ester with an oxidizing agent to form a diol, then protecting a with a diol functional group 2308 on said diol to form a protected diol 2310. Deprotecting the protected ester group on said protected diol to produce an acid precursor, and then performing a coupling on said acid precursor in the presence of a vinyl amine derivative 2312 to form an intermediate functionalized-protected diol. Selectively deprotecting at the N-terminus of said intermediate functionalized-protected diol, followed by conducting a coupling of said intermediate functionalized-protected diol with a functionalized amino acid 2316 to produce a functionalized-protected diol 2318. Treating said functionalized-protected diol with an oxidizing agent to produce an RCM (Ring Closing Metathesis) precursor 2320. Then performing a ring-closing operation on said RCM precursor using catalyst produces a proteasome-inhibiting core precursor 2322, and reducing said proteasome-inhibiting core precursor to obtain said proteasome inhibiting core 2324.

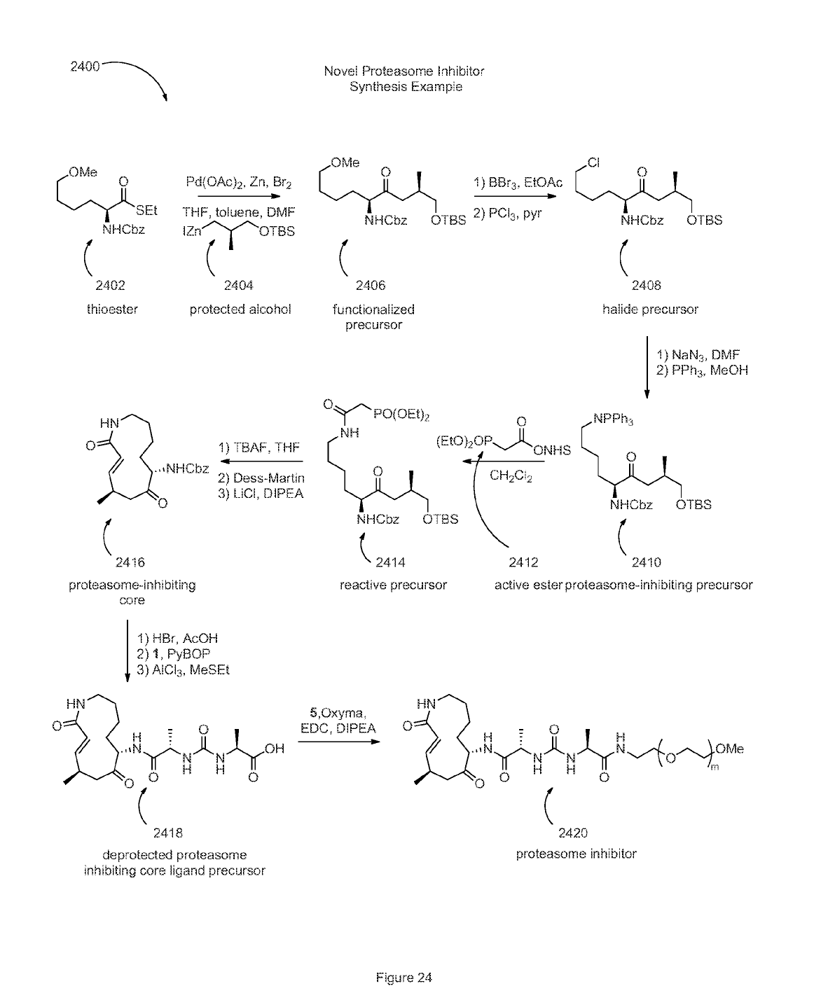

Scheme 2400, in accordance with several embodiments of the present invention, depicts synthesis of additional proteasome inhibitors, as shown in FIG. 24. This scheme includes using a thioester 2402 which can be coupled together with protected alcohol 2404 to provide functionalized precursor 2406 by Fukuyama conditions. A cross-metathesis reaction with a 1-butene derivative and Grubbs II catalyst provides a halide precursor 2408. By performing a nucleophilic substitution on the halide of 2408 with sodium azide followed by the Staudinger reaction prepares proteasome-inhibiting precursor 2410. This is coupled with the phosphonoacetic acid active ester 2412 which provides the precursor to the Horner-Wadsworth-Emmons reaction (reactive precursor 2414). After deprotection of the alcohol it can be oxidized to the aldehyde using Dess-Martin conditions followed by a HWE cyclization to create proteasome inhibiting core 2416. Furthermore the scheme describes one of the possible ligand couplings to said proteasome inhibiting core 2416 continuing with the deprotection of the proteasome inhibiting core 2416 to provide a free amine proteasome inhibiting core which is coupled with compound 1 to provide proteasome inhibiting core ligand precursor. Removal of the protecting group on proteasome inhibiting core ligand precursor provides deprotected proteasome inhibiting core ligand precursor 2418. Then coupling on compound 5 is performed to produce the final proteasome inhibiting core with ligand 2420.

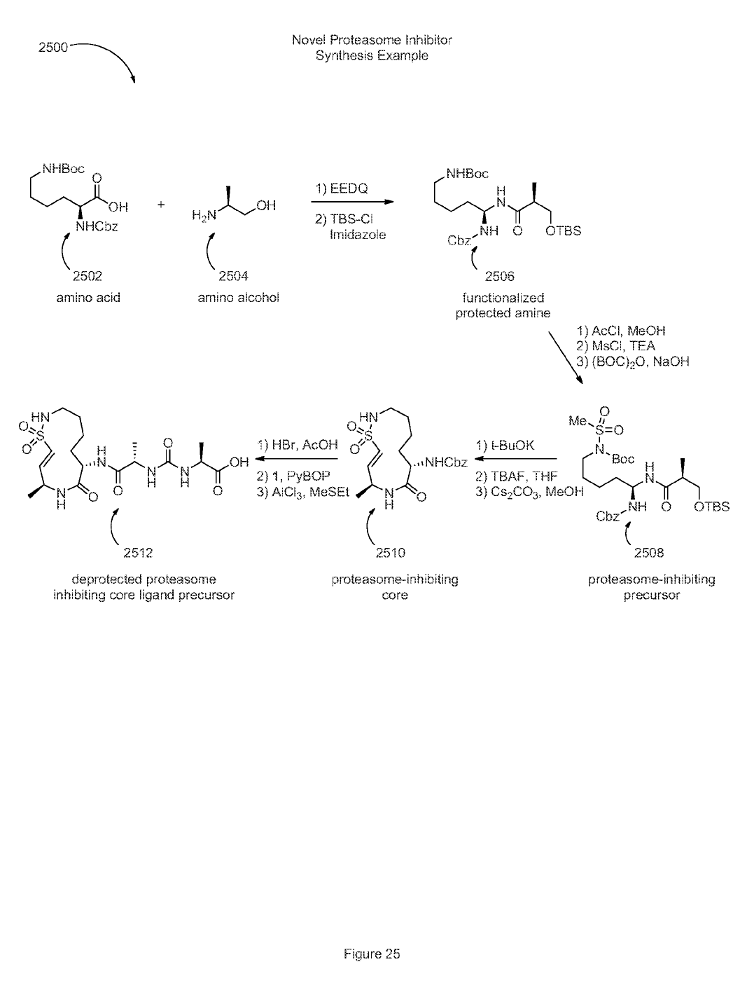

Scheme 2500, in accordance with several embodiments of the present invention, depicts synthesis of an additional proteasome inhibiting core-ligand precursor, as shown in FIG. 25. This scheme includes using an amino acid 2502 and an amino alcohol 2504, combined by a peptide coupling reaction then an alcohol protection, which produces functionalized protected amine 2506. Deprotecting the amine group on said functionalized protected amine produces a functionalized amine compound. Attaching a sulfone group to said functionalized amine compound produces a sulfone compound. Re-protecting the amine on the sulfone compound produces a proteasome inhibiting precursor. Upon addition of a strong base followed by deprotection of the primary alcohol, and finally a reducing agent such as caesium carbonate, proteasome inhibiting core 2510 is produced. Furthermore the scheme describes one of the possible ligand couplings to said proteasome inhibiting core 2510 continuing with the deprotection of the proteasome inhibiting core 2510 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2512. Further steps may be performed to provide desired ligand on said proteasome inhibiting core.

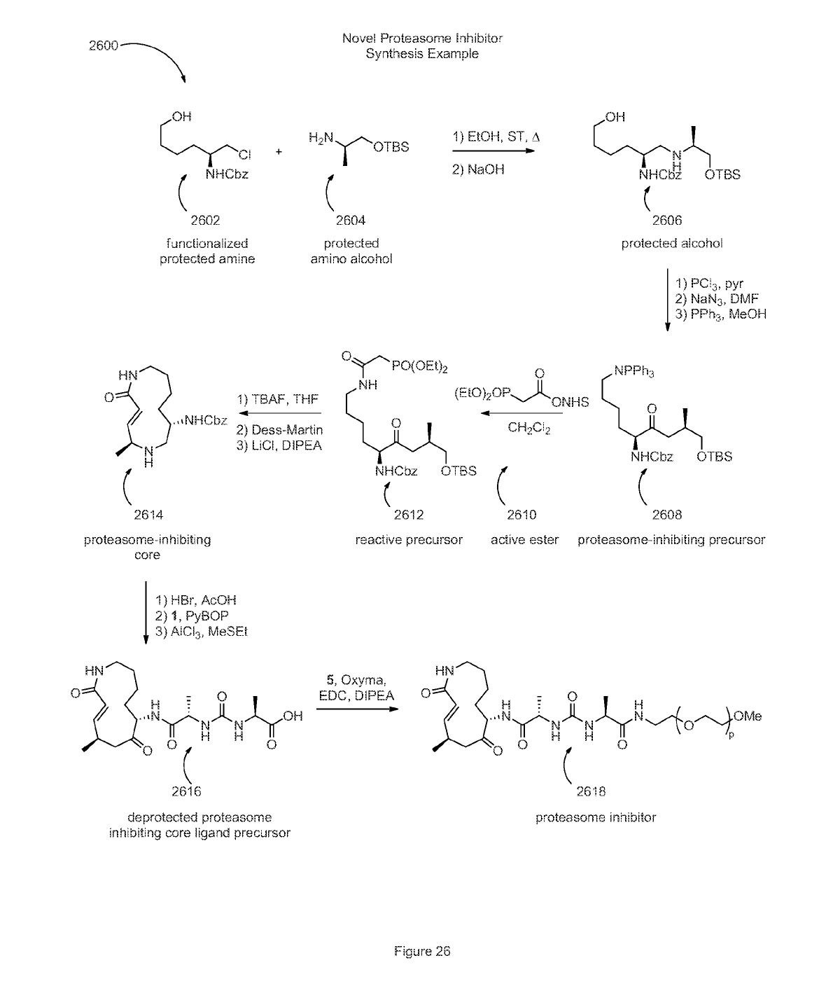

Scheme 2600, in accordance with several embodiments of the present invention, depicts synthesis of additional proteasome inhibitors, as shown in FIG. 26. This scheme includes using a functionalized protected amine 2602 and an amino alcohol 2604, combined by a nucleophilic substitution reaction, which produces protected alcohol 2606. Halogenating said protected alcohol compound to obtain a halide compound, then performing a nucleophilic substitution on the halide with sodium azide, followed by the Staudinger reaction, prepares proteasome inhibiting precursor 2608. This is coupled with the phosphonoacetic acid active ester 2610, which provides the precursor to the Horner-Wadsworth-Emmons reaction (reactive precursor 2612). After deprotection of the alcohol, it can be oxidized to the aldehyde using Dess-Martin conditions followed by a HWE cyclization, which provides proteasome inhibiting core 2614. Furthermore the scheme describes one of the possible ligand couplings to said proteasome inhibiting core 2614, continuing with the deprotection of the proteasome inhibiting core 2614 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2616. Then coupling on compound 5 is performed to produce the final proteasome inhibiting core with ligand 2618.

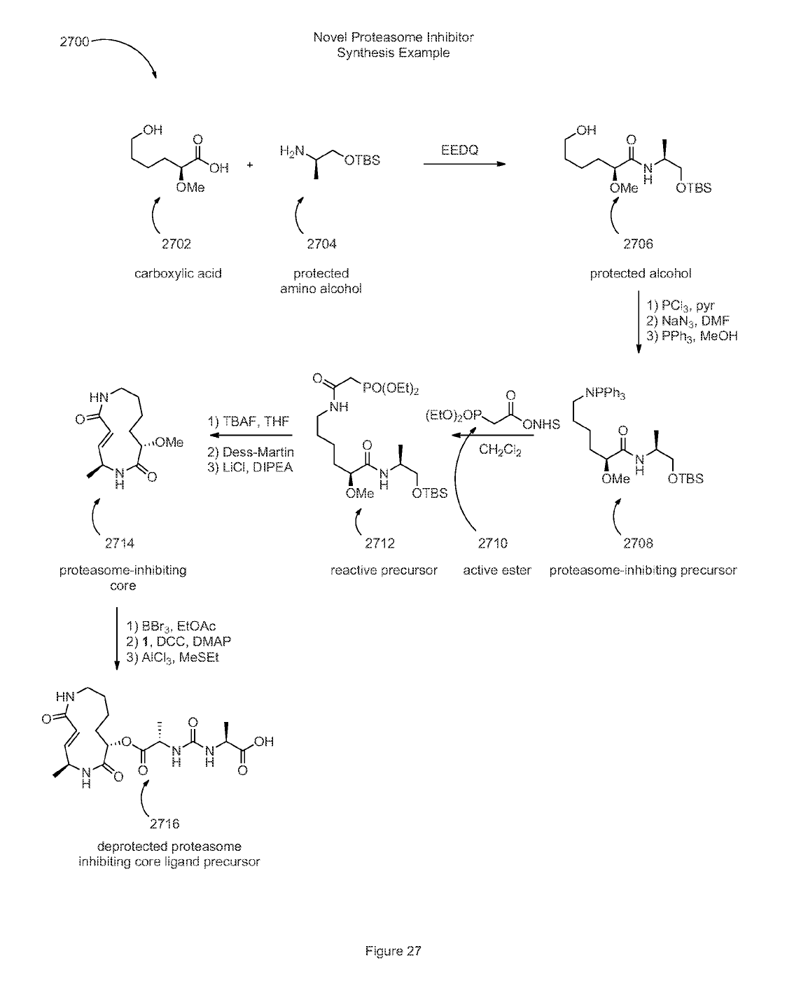

Scheme 2700, in accordance with several embodiments of the present invention, depicts synthesis of a proteasome inhibiting core-ligand precursor, as shown in FIG. 27. This scheme includes using a carboxylic acid 2702 and an amino alcohol 2704, combined by a peptide coupling reaction to produce protected alcohol 2706. Halogenating said protected alcohol compound to obtain a halide compound, then performing a nucleophilic substitution on the halide with sodium azide, followed by the Staudinger reaction, prepares proteasome inhibiting precursor 2708. This is coupled with phosphonoacetic acid active ester 2710, which provides the precursor to the Horner-Wadsworth-Emmons reaction (reactive precursor 2712). This then can be oxidized to the aldehyde using Dess-Martin conditions followed by a HWE cyclization to produce proteasome inhibiting core 2714. Furthermore the scheme describes one of the possible ligand couplings to said proteasome inhibiting core 2714, continuing with the deprotection of the proteasome inhibiting core 2714 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2716. Further steps may be performed to provide desired ligand on said proteasome inhibiting core.

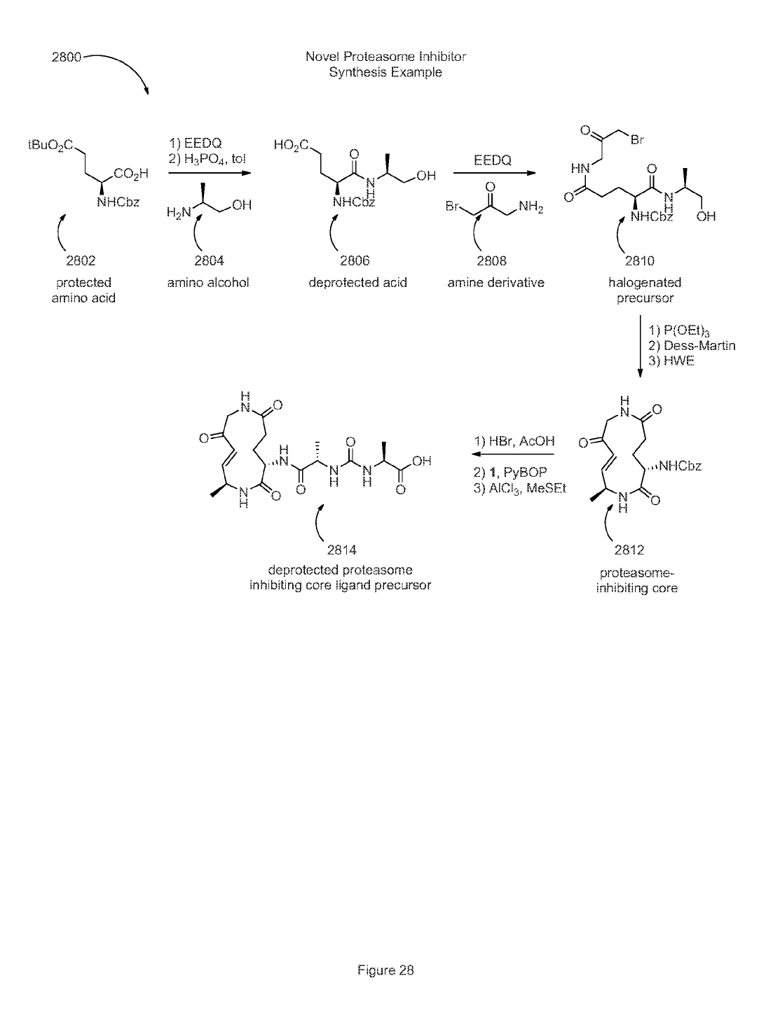

Scheme 2800, in accordance with several embodiments of the present invention, depicts synthesis of an additional proteasome inhibiting core-ligand precursor, as shown in FIG. 28. This scheme includes using a protected amino acid 2802 and an amino alcohol 2804, combined by a peptide coupling reaction to produce a protected acid. After deprotection of the remaining carboxyl group (2806), a second coupling reaction is carried out with said deprotected acid 2806 and amine derivative 2808 to produce halogenated precursor 2810. Substituting an active group for a halogenated site on said halogenated precursor forms an HWE reaction precursor. Then oxidizing the HWE reaction precursor to yield an aldehyde-based proteasome inhibiting precursor followed by a HWE cyclization to produce proteasome inhibiting core 2812. Furthermore the scheme describes one of the possible ligand couplings to said proteasome inhibiting core 2812 continuing with the deprotection of the proteasome inhibiting core 2812 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2814. Further steps may be performed to provide a desired ligand on the proteasome inhibiting core.

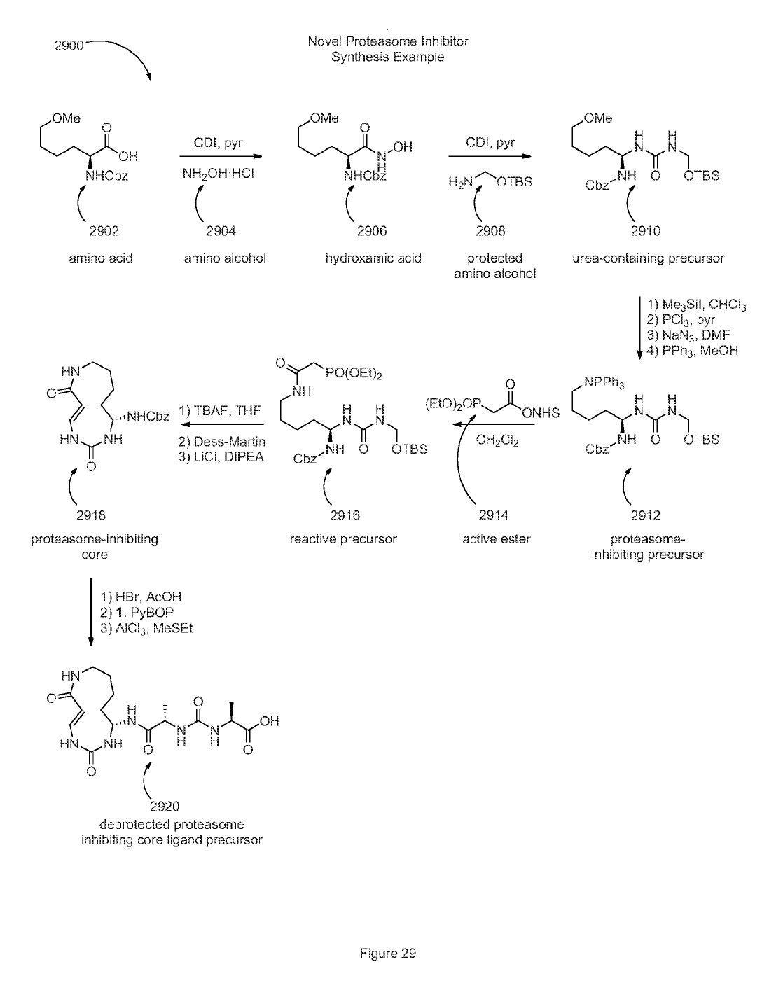

Scheme 2900, in accordance with several embodiments of the present invention, depicts synthesis of an additional proteasome inhibiting core-ligand precursor, as shown in FIG. 29. This scheme includes using an amino acid 2902, Carbonyldiimidazole, pyridine, and Hydroxylamine hydrochloride to synthesize hydroxamic acid 2904, which can be retreated with Carbonyldiimidazole, pyridine, and a protected amino alcohol 2908, to synthesize the urea containing precursor 2910. Deprotecting the alcohol component of said urea-containing precursor forms the deprotected urea-containing precursor. Halogenating said deprotected urea-containing precursor with active halogenating agent obtains a halide compound. To introduce the last nitrogen, a nucleophilic substitution on the halide with sodium azide followed by the Staudinger reaction prepares proteasome inhibiting precursor 2912. This is coupled with the phosphonoacetic acid active ester 2914, which provides the precursor to the Horner-Wadsworth-Emmons reaction (reactive precursor 2916). After deprotection of the alcohol, it can be oxidized to the aldehyde using Dess-Martin conditions, followed by a HWE cyclization, to produce proteasome inhibiting core 2918. Furthermore the scheme describes one of the possible ligand couplings to said proteasome inhibiting core 2918, continuing with the deprotection of the proteasome inhibiting core 2918 to provide a free amine proteasome inhibiting core, which is coupled with compound 1 to provide proteasome inhibiting core ligand precursor. Removal of the protecting group on proteasome inhibiting core ligand precursor provides deprotected proteasome inhibiting core ligand precursor 2920. Further steps may be performed to provide a desired ligand on said proteasome inhibiting core.

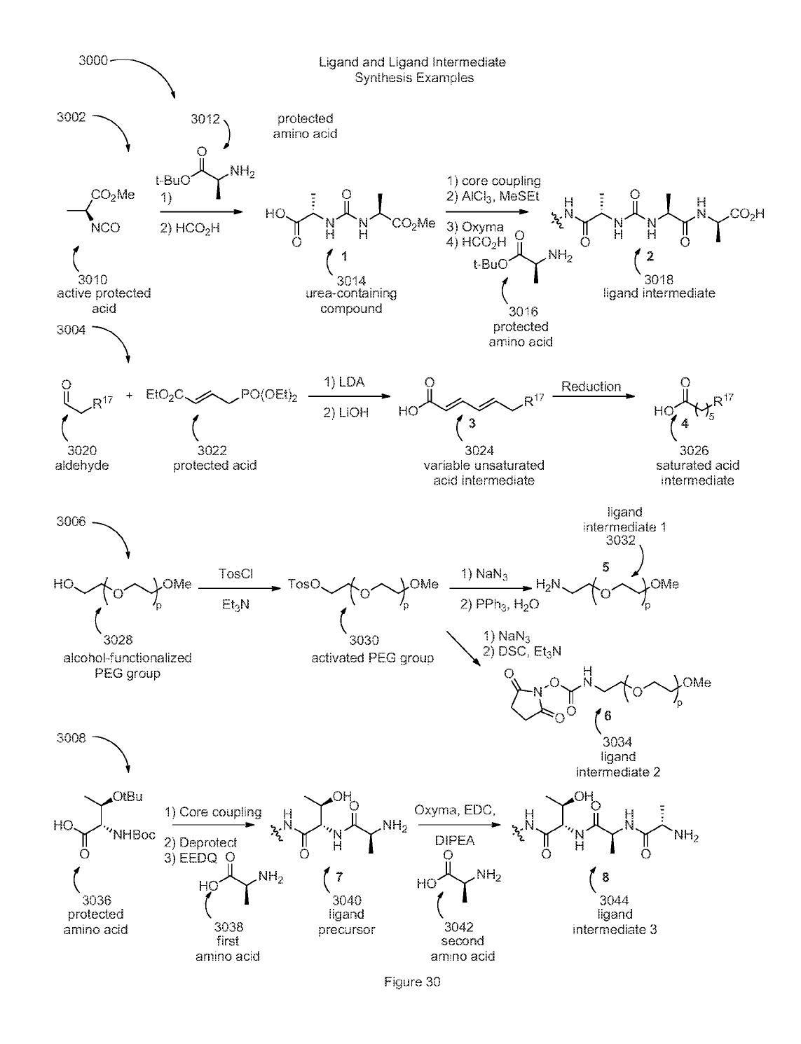

Scheme 3000, in accordance with several embodiments of the present invention, depicts synthesis of various embodiments of ligands and ligand intermediates for synthesizing possible ligand materials for coupling reactions, as shown in FIG. 30.

Scheme 3002, in accordance with several embodiments of the present invention, depicts one embodiment of formation of a urea containing ligand while coupling another amino acid to extend said ligand. L-alanine-derived isocyanate 3010 is reacted with L-alanine tert-butyl ester 3012, which is subsequently deprotected to form the bis(alanine)urea 1 (3014), also referred to as urea-containing compound 1. The urea-containing compound coupled with a core and subsequently deprotected primes a peptide coupling with L-alanine tert-butyl ester 3016, followed by a final deprotection, to provide compound 2 (3018), as shown in FIG. 30.

Scheme 3004, in accordance with several embodiments of the present invention, depicts one embodiment of a synthesis route to a partly saturated and/or unsaturated carboxylic acid by the HWE reaction, followed by a possible reduction. Starting with an aldehyde 3020, a HWE reaction with triethyl-4-phosphono crotonate 3022, followed by a deprotection, is performed to provide a variable saturated acid intermediate 3. If desired, a reduction can be performed with Pd/C and hydrogen gas to obtain the saturated acid intermediate 4 (3026), as shown in FIG. 30.

Scheme 3006, in accordance with several embodiments of the present invention, depicts the steps (according to certain embodiments) to add a PEG group onto either a carboxylic acid or an amine. These are provided as an example and are in no way intended to be limiting. To prepare the PEG group, one could start with triethylene glycol monomethyl ether 3028 (for n=2), which will react with tosyl chloride under basic conditions to form a tosylated alcohol 3030. A nucleophilic displacement with an azide salt and subsequent triphenylphosphane-mediated reduction leads to amine 5 3032, also referred to as ligand intermediate 1. From reaction intermediate 3030 a nucleophilic displacement with an azide salt followed by disuccinimidyl carbonate and triethylamine results in the PEG succinimidyl carbamate 6 (3034), as shown in FIG. 30.

Scheme 3008 in accordance with several embodiments of the present invention, depicts steps to synthesize one embodiment of a peptide ligand. First, a protected amino acid 3036 is coupled to a proteasome inhibitor core, followed by a deprotection and coupling of first amino acid 3038 to obtain ligand precursor 3040. Another coupling with second amino acid 3042 is performed to obtain ligand intermediate 3, as shown in FIG. 30.

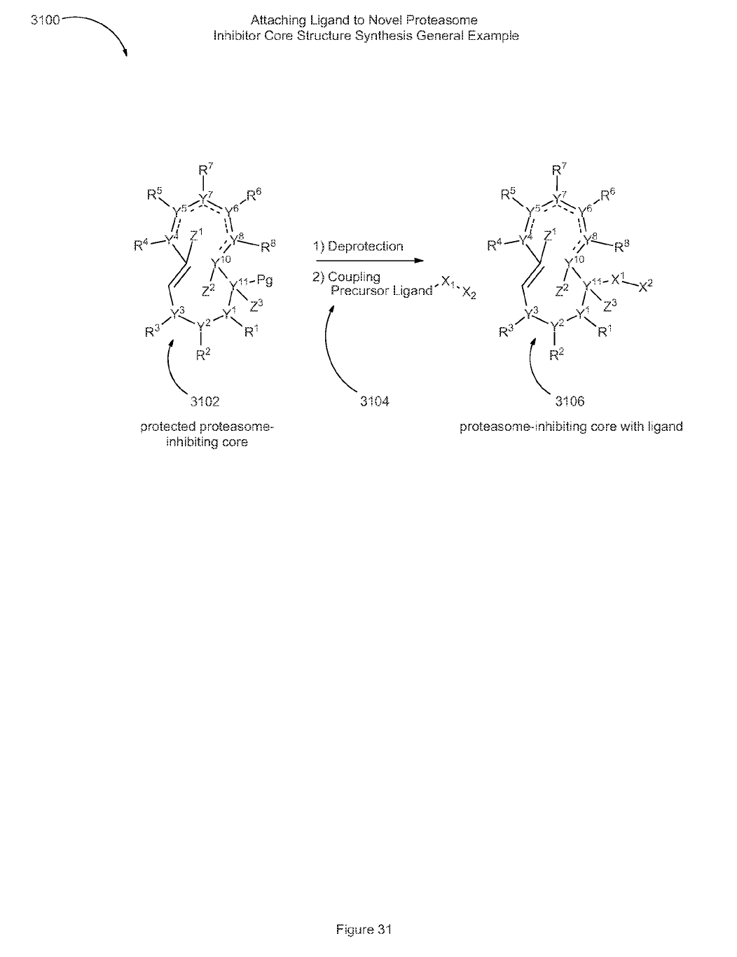

Scheme 31, in accordance with several embodiments of the present invention, depicts one embodiment of a method of attaching a ligand to novel proteasome inhibitor core, as shown in FIG. 31. The protected proteasome inhibitor core 3102 with the protecting group shown as (Pg) is deprotected, and then coupled with ligand precursor 3104, producing proteasome inhibitor core with ligand 3106.

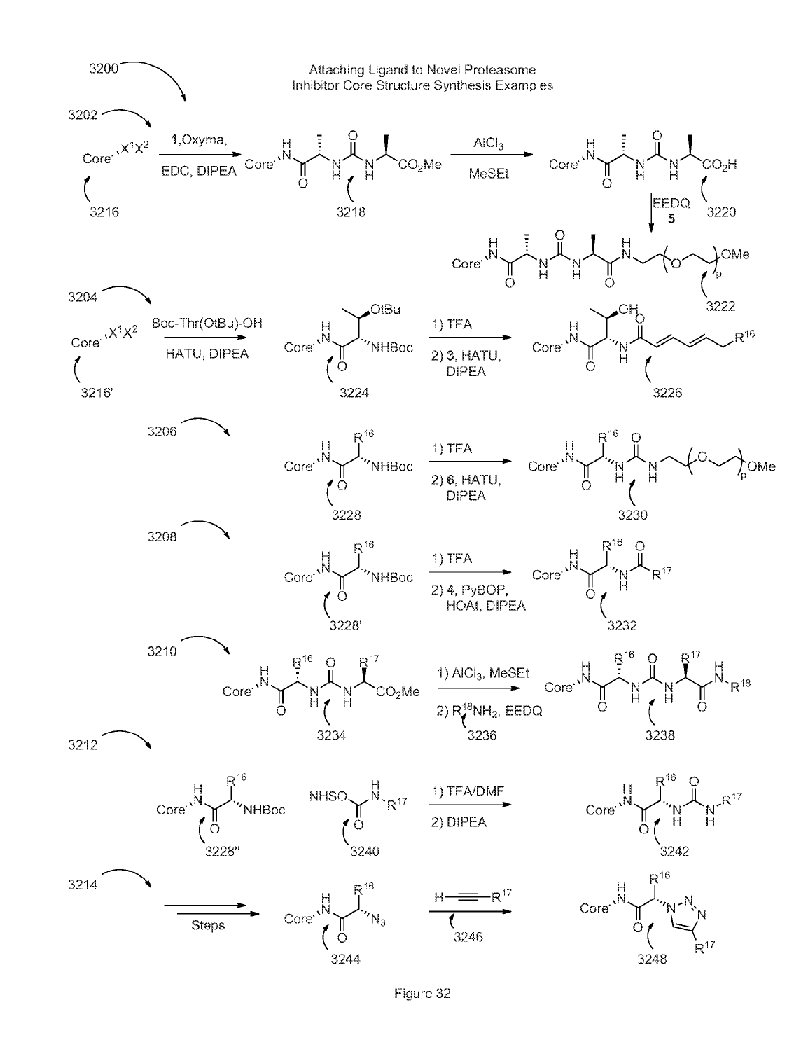

Scheme 32, in accordance with several embodiments of the present invention, depicts one embodiment of methods for attaching ligands to novel proteasome inhibitor core structures, as shown in FIG. 32. This scheme includes several examples for coupling side chain ligands to core structures. For simplicity of these examples, X1 is represented as an amine.

Scheme 3202, in accordance with several embodiments of the present invention, depicts one embodiment of a peptide coupling with one of the core structures, 3216, and urea-containing compound 1 (FIG. 28). The proteasome inhibitor core with protected ligand 3018 can be deprotected, priming a peptide coupling with amine compound 5, which provides a pegylated urea side chain attached to any specified core 3222.

Scheme 3204, in accordance with several embodiments of the present invention, depicts an additional embodiment of a peptide coupling with core structure 3216′ and with a protected threonine amino acid, to obtain intermediate 3224. After deprotection, a second peptide coupling is done with variable saturated acid 3 (FIG. 28), to synthesize a lipophilic side chain with an amino acid attached to any specified core 3226.

Scheme 3206, in accordance with several embodiments of the present invention, depicts one embodiment of the deprotection of core intermediate 3228, followed by a coupling reaction with PEG succinimidyl carbamate 6, to afford pegylated urea side chain attached to any specified core 3230.

Scheme 3208, in accordance with several embodiments of the present invention, depicts one embodiment of the deprotection of nitrogen, which is attached to core intermediate 3228′, followed by a peptide coupling with a variable defined carboxylic acid 4 to extend the side chain to afford a varied amino acid side chain attached to any specified core 3232.

Scheme 3210, in accordance with several embodiments of the present invention, depicts one embodiment of the deprotection of a carboxyl group, which is attached to core intermediate 3234, followed by a peptide coupling with an amine 3236 to extend the side chain to afford a varied amino acid side chain attached to any specified core 3238.

Scheme 3212, in accordance with several embodiments of the present invention, depicts a Boc protected amino acid attached to any specified core 3228, for which a deprotection can be done, followed by a coupling reaction with variable defined succinimidyl carbamate 3240 to afford a varied urea containing side chain attached to any specified core 3242.

Scheme 3214, in accordance with several embodiments of the present invention, depicts one embodiment of the pre-constructed core with an azide group 3244 on the side chain to provide a triazole 3248 through ‘click’ chemistry conditions with a terminal alkyne 3246.

Although the embodiments of the inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. For all of the embodiments described herein the steps of the methods need not be performed sequentially. Thus, it is intended that the scope of the present inventions herein disclosed should not be limited by the particular disclosed embodiments described above.

The ranges disclosed herein also encompass any and all overlap, subranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

1. A proteasome inhibitor comprising:
a core ring structure selected from a group consisting of a first structure, a second structure and a third structure, and said first structure is represented by Formula I, which is: 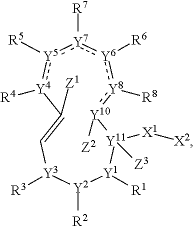
said second structure is represented by Formula II, which is: 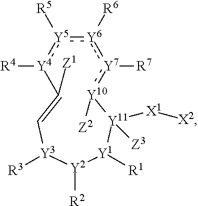
said third structure is represented by Formula III, which is: 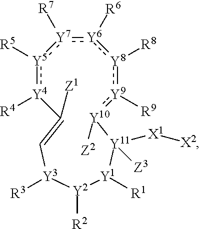
and
wherein Y¹ is at least one member selected from a group consisting of nitrogen, NH, oxygen, OH, sulfur, SO, SO₂, and carbon;
wherein each of Y², Y⁴, and Y⁶ is at least one member selected from a group consisting of nitrogen, NH, oxygen, OH, sulfur, SO, SO₂, CO, and carbon;
wherein X¹ is absent or at least one member selected from a group consisting of hydrogen, OH, CH₂O, COH, CO₂H, halide, NH, S, P(X²)₃, BOH, B(OH)₂, aryl, carbocycle, substituted aryl, substituted carbocycle, heterocycle, substituted heterocycle, alkyl, substituted alkyl, alkenyl, alkenyl substituted, alkynyl, alkynyl substituted, aralkyl, (CH₂CH₂Y¹³)ᵣ, JAJ, an amino-acid-based moiety, and (Y²R¹⁰LQR¹¹)_{q}, and each of q and r is an integer value between 1 and 10;
wherein each of Y³, Y⁵, Y⁷, Y⁸, Y⁹, Y¹⁰, Y¹¹, Y¹², and Y¹³ is a moiety; and
wherein each of X², R¹, R², R³, R⁴, R⁵, R⁶, R⁷, R⁸, R⁹, R¹⁰, R¹¹, Z¹, Z², Z³, A, J, L, and Q is a moiety or absent.

2.-436. (canceled)
